(19) United States
(12) Patent Application Publication
(10) Pub. No.: US 2015/0166520 A1
(43) Pub. Date: Jun. 18, 2015

(54) AMIDO-SUBSTITUTED PYRIMIDINONE DERIVATIVES USEFUL FOR THE TREATMENT OF HIV INFECTION

(71) Applicant: MERCK SHARP & DOHME CORP., Rahway, NJ (US)

(72) Inventors: Paul J. Coleman, Harleysville, PA (US); Abbas M. Walji, Lansdale, PA (US); Qun Dang, Westfield, NJ (US); David Jonathan Bennett, New Hope, PA (US); Sophie D. Clas, Montreal (CA); John S. Wai, Harleysville, PA (US); Jaume Balsells-Padros, Upper Gwynedd, PA (US); Henry Y. Wu, North Wales, PA (US); Ronald L. Smith, Yardley, PA (US); Rebecca Nofsinger, Lansdale, PA (US); Rosa I. Sanchez, Audubon, PA (US)

(73) Assignee: MERCK SHARP & DOHME CORP., Rahway, NJ (US)

(21) Appl. No.: 14/415,885

(22) PCT Filed: Jul. 16, 2013
(86) PCT No.: PCT/US13/50715

§ 371 Date: Jan. 20, 2015

Related U.S. Application Data
(60) Provisional application No. 61/673,886, filed on Jul. 20, 2012.

(51) Int. Cl. C07D 413/12; A61K 45/06; A61K 31/513

(52) U.S. Cl. CPC C07D 413/12; A61K 31/513; A61K 45/06

(57) ABSTRACT

The present invention relates to Amido-Substituted Pyrimidinone Derivatives of Formula (I), and pharmaceutically acceptable salts thereof, wherein R¹, R², R³, R⁴, R⁵ and R⁶ are as defined herein. The present invention also relates to compositions comprising at least one Amido-Substituted Pyrimidinone Derivative, and methods of using the Amido-Substituted Pyrimidinone Derivatives for treating or preventing HIV infection in a subject.

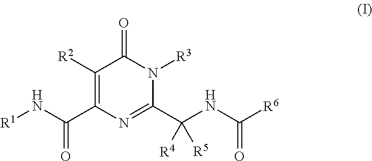

### FIELD OF THE INVENTION

The present invention relates to Amido-Substituted Pyrimidinone Derivatives, compositions comprising at least one Amido-Substituted Pyrimidinone Derivative, and methods of using the Amido-Substituted Pyrimidinone Derivatives for treating or preventing HIV infection in a subject.

### BACKGROUND OF THE INVENTION

A retrovirus designated human immunodeficiency virus (HIV), particularly the strains known as HIV type-1 (HIV-1) virus and type-2 (HIV-2) virus, is the etiological agent of the complex disease that includes progressive destruction of the immune system (acquired immune deficiency syndrome; AIDS) and degeneration of the central and peripheral nervous system. A common feature of retrovirus replication is the insertion by virally-encoded integrase of +proviral DNA into the host cell genome, a required step in HIV replication in human T-lymphoid and monocytoid cells. Integration is believed to be mediated by integrase in three steps: assembly of a stable nucleoprotein complex with viral DNA sequences; cleavage of two nucleotides from the 3′ termini of the linear proviral DNA; covalent joining of the recessed 3′ OH termini of the proviral DNA at a staggered cut made at the host target site. The fourth step in the process, repair synthesis of the resultant gap, may be accomplished by cellular enzymes.

Nucleotide sequencing of HIV shows the presence of a pol gene in one open reading frame [Ratner, L. et al., Nature, 313, 277(1985)] Amino acid sequence homology provides evidence that the pol sequence encodes reverse transcriptase, integrase and an HIV protease [Toh, H. et al., EMBO J. 4, 1267 (1985); Power, M. D. et al., Science, 231, 1567 (1986); Pearl, L. H. et al., Nature, 329, 351 (1987)]. All three enzymes have been shown to be essential for the replication of HIV.

It is known that some antiviral compounds which act as inhibitors of HIV replication are effective agents in the treatment of AIDS and similar diseases, including reverse transcriptase inhibitors such as azidothymidine (AZT) and efavirenz and protease inhibitors such as indinavir and nelfinavir. The compounds of this invention are inhibitors of HIV integrase and inhibitors of HIV replication. The inhibition of integrase in vitro and HIV replication in cells is a direct result of inhibiting the strand transfer reaction catalyzed by the recombinant integrase in vitro in HIV infected cells.

The following references are of interest as background:

International Publication Nos. WO 11/045330 and WO 11/121105 disclose macrocyclic compounds having HIV integrase inhibitory activity.

Kinzel et al., Tet. Letters 2007, 48(37): pp. 6552-6555 discloses the synthesis of tetrahydropyridopyrimidones as a scaffold for HIV-1 integrase inhibitors.

Ferrara et al., Tet. Letters 2007, 48(37), pp. 8379-8382 discloses the synthesis of a hexahydropyrimido[1,2-a]azepine-2-carboxamide derivative useful as an HIV integrase inhibitor.

Muraglia et al., J. Med. Chem. 2008, 51: 861-874 discloses the design and synthesis of bicyclic pyrimidinones as potent and orally bioavailable HIV-1 integrase inhibitors.

US2004/229909 discloses certain compounds having integrase inhibitory activity.

U.S. Pat. No. 7,232,819 and US Patent Publication No. US 2007/0083045 disclose certain 5,6-dihydroxypyrimidine-4-carboxamides as HIV integrase inhibitors.

U.S. Pat. No. 7,169,780, U.S. Pat. No. 7,217,713, and US Patent Publication No. US 2007/0123524 disclose certain N-substituted 5-hydroxy-6-oxo-1,6-dihydropyrimidine-4-carboxamides as HIV integrase inhibitors.

U.S. Pat. No. 7,279,487 discloses certain hydroxynaphthyridinone carboxamides that are useful as HIV integrase inhibitors.

U.S. Pat. No. 7,135,467 and U.S. Pat. No. 7,037,908 disclose certain pyrimidine carboxamides that are useful as HIV integrase inhibitors.

U.S. Pat. No. 7,211,572 discloses certain nitrogenous condensed ring compounds that are HIV integrase inhibitors.

U.S. Pat. No. 7,414,045 discloses certain tetrahydro-4H-pyrido[1,2-a]pyrimidine carboxamides, hexahydropyrimido[1,2-a]azepine carboxamides, and related compounds that are useful as HIV integrase inhibitors.

International Publication No. WO 2006/103399 discloses certain tetrahydro-4H-pyrimidooxazepine carboxamides, tetrahydropyrazinopyrimidine carboxamides, hexahydropyrimidodiazepine carboxamides, and related compounds that are useful as HIV integrase inhibitors.

US Patent Publication No. US 2007/0142635 discloses processes for preparing hexahydropyrimido[1,2-a]azepine-2-carboxylates and related compounds.

US Patent Publication No. US 2007/0149556 discloses certain hydroxypyrimidinone derivatives having HIV integrase inhibitory activity.

Various pyrimidinone compounds useful as HIV integrase inhibitors are also disclosed in U.S. Pat. No. 7,115,601, U.S. Pat. No. 7,157,447, U.S. Pat. No. 7,173,022, U.S. Pat. No. 7,176,196, U.S. Pat. No. 7,192,948, U.S. Pat. No. 7,273,859, and U.S. Pat. No. 7,419,969.

US Patent Publication No. US 2007/0111984 discloses a series of bicyclic pyrimidinone compounds useful as HIV integrase inhibitors.

US Patent Publication Nos. US 2006/0276466, US 2007/0049606, US 2007/0111985, US 2007/0112190, US 2007/0281917 and US 2008/0004265 each disclose a series of bicyclic pyrimidinone compounds useful as HIV integrase inhibitors.

International Publication No. WO 12/009446 discloses a series of novel pyrimidine carboxamide derivatives useful as HIV integrase inhibitors.

Raltegravir (N-[(4-fluorophenyl)methyl]-1,6-dihydro-5-hydroxy-1-methyl-2-[1-methyl-1-[[(5-methyl-1,3,4-oxadiazol-2-yl)carbonyl]amino]ethyl]-6-oxo-4-pyrimidinecarboxamide and N-(2-(4-(4-fluorobenzylcarbamoyl)-5-hydroxy-1-methyl-6-oxo-1,6-dihydropyrimidin-2-yl)propan-2-yl)-5-methyl-1,3,4-oxadiazole-2-carboxamide) is an approved drug for the treatment of AIDS. Raltegravir inhibits the activity of HIV-1 integrase, including strains that are resistant to other anti-retroviral drugs.

### SUMMARY OF THE INVENTION

In one aspect, the present invention provides Compounds of Formula (I):

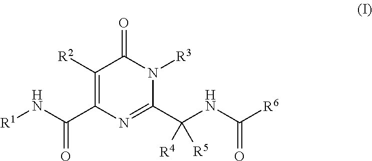

and pharmaceutically acceptable salts thereof,
wherein:

R¹ is selected from —(C₁-C₆ alkylene)-(C₆-C₁₀ aryl), —(C₁-C₆ alkylene)-(5 or 6-membered monocyclic heteroaryl) or —(C₁-C₆ alkylene)-(9 or 10-membered bicyclic heteroaryl, wherein said C₆-C₁₀ aryl group, said 5 or 6-membered monocyclic heteroaryl group and said 9 or 10-membered bicyclic heteroaryl group can be optionally substituted with up to four R⁷ groups, which can be the same or different;

R² is —O—(C₁-C₆ alkylene)-X—C(O)—Y—R⁹;

R³ is H or C₁-C₆ alkyl;

R⁴ is H or C₁-C₆ alkyl;

R⁵ is H or C₁-C₆ alkyl;

R⁶ is selected from C₆-C₁₀ aryl, 5 or 6-membered monocyclic heteroaryl and 9 or 10-membered bicyclic heteroaryl, any of which can be optionally substituted with up to four R⁷ groups;

R⁷ is C₁-C₆ alkyl, 5 or 6-membered heterocycloalkyl, C₁-C₆ hydroxyalkyl, C₁-C₆ haloalkyl, halo, —CN, —N(R⁸)₂, —CH₂N(R⁸)₂, —OR⁸, —C(O)OR⁸, —SR⁸, —S(O)₂R⁸ or —C(O)N(R⁸)₂, wherein said 5 or 6-membered heterocycloalkyl group can be optionally substituted with a group selected from C₁-C₆ alkyl, halo, C₁-C₆ hydroxyalkyl, C₁-C₆ haloalkyl, halo, —CN, —N(R⁸)₂ and —OR⁸;

each occurrence of R⁸ is independently H or C₁-C₆ alkyl;

R⁹ is selected from C₁-C₆ alkyl, C₆-C₁₀ aryl, 5 or 6-membered monocyclic heteroaryl, 9 or 10-membered bicyclic heteroaryl, 3 to 7-membered monocyclic heterocycloalkyl, 8 to 10-membered bicyclic heterocycloalkyl and C₃-C₆ cycloalkyl, wherein said C₁-C₆ alkyl group can be optionally substituted with R¹⁰, and wherein said C₆-C₁₀ aryl group, 5 or 6-membered monocyclic heteroaryl group, 9 or 10-membered bicyclic heteroaryl group, 3 to 7-membered monocyclic heterocycloalkyl group, 8 to 10-membered bicyclic heterocycloalkyl group and C₃-C₆ cycloalkyl group can be optionally substituted with up to four R⁷ groups, which can be the same or different;

R¹⁰ is selected from C₆-C₁₀ aryl, 5 or 6-membered monocyclic heteroaryl, 9 or 10-membered bicyclic heteroaryl, 3 to 7-membered monocyclic heterocycloalkyl, 8 to 10-membered bicyclic heterocycloalkyl and C₃-C₆ cycloalkyl, wherein said C₆-C₁₀ aryl group, 5 or 6-membered monocyclic heteroaryl group, 9 or 10-membered bicyclic heteroaryl group, 3 to 7-membered monocyclic heterocycloalkyl group, 8 to 10-membered bicyclic heterocycloalkyl group and C₃-C₆ cycloalkyl group can be optionally substituted with up to four R⁷ groups, which can be the same or different;

X is O or NH; and

Y is a bond, O or NH.

The Compounds of Formula (I) (also referred to herein as the “Amido-Substituted Pyrimidinone Derivatives”) and pharmaceutically acceptable salts thereof can be useful, for example, for inhibiting HIV viral replication or replicon activity, and for treating or preventing HIV infection in a subject.

As illustrated below, the R² group of the Compounds of Formula (I) can be hydrolyzed under physiological conditions to provide the corresponding hydroxy compounds.

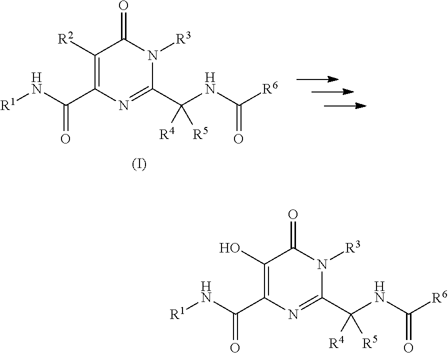

Accordingly, the present invention provides methods for treating or preventing HIV infection in a subject, comprising administering to the subject an effective amount of at least one Amido-Substituted Pyrimidinone Derivative.

The details of the invention are set forth in the accompanying detailed description below.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### DETAILED DESCRIPTION OF THE INVENTION

The present invention relates to Amido-Substituted Pyrimidinone Derivatives, compositions comprising at least one Amido-Substituted Pyrimidinone Derivative, and methods of using the Amido-Substituted Pyrimidinone Derivatives for treating or preventing HIV infection in a subject.

### DEFINITIONS AND ABBREVIATIONS

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “—O-alkyl,” etc. . . .

As used herein, and throughout this disclosure, the following terms, unless otherwise indicated, shall be understood to have the following meanings:

A “subject” is a human or non-human mammal. In one embodiment, a subject is a human. In another embodiment, a subject is a primate. In another embodiment, a subject is a monkey. In another embodiment, a subject is a chimpanzee. In still another embodiment, a subject is a rhesus monkey.

The term “effective amount” as used herein, refers to an amount of Amido-Substituted Pyrimidinone Derivative and/or an additional therapeutic agent, or a composition thereof that is effective in producing the desired therapeutic, ameliorative, inhibitory or preventative effect when administered to a subject suffering from HIV infection or AIDS. In the combination therapies of the present invention, an effective amount can refer to each individual agent or to the combination as a whole, wherein the amounts of all agents administered are together effective, but wherein the component agent of the combination may not be present individually in an effective amount.

The term “preventing,” as used herein with respect to an HIV viral infection or AIDS, refers to reducing the likelihood or severity of HIV infection or AIDS.

The term “alkyl,” as used herein, refers to an aliphatic hydrocarbon group having one of its hydrogen atoms replaced with a bond. An alkyl group may be straight or branched and contain from about 1 to about 20 carbon atoms. In one embodiment, an alkyl group contains from about 1 to about 12 carbon atoms. In different embodiments, an alkyl group contains from 1 to 6 carbon atoms (C₁-C₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. An alkyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. In one embodiment, an alkyl group is linear. In another embodiment, an alkyl group is branched. Unless otherwise indicated, an alkyl group is unsubstituted.

The term “alkenyl,” as used herein, refers to an aliphatic hydrocarbon group containing at least one carbon-carbon double bond and having one of its hydrogen atoms replaced with a bond. An alkenyl group may be straight or branched and contain from about 2 to about 15 carbon atoms. In one embodiment, an alkenyl group contains from about 2 to about 12 carbon atoms. In another embodiment, an alkenyl group contains from about 2 to about 6 carbon atoms. Non-limiting examples of alkenyl groups include ethenyl, propenyl, n-butenyl, 3-methylbut-2-enyl, n-pentenyl, octenyl and decenyl. An alkenyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)-alkyl, —O—C(O)-aryl, —O—C(O)-cycloalkyl, —C(O)OH and —C(O)O-alkyl. The term “C₂-C₆ alkenyl” refers to an alkenyl group having from 2 to 6 carbon atoms. Unless otherwise indicated, an alkenyl group is unsubstituted.

The term “alkynyl,” as used herein, refers to an aliphatic hydrocarbon group containing at least one carbon-carbon triple bond and having one of its hydrogen atoms replaced with a bond. An alkynyl group may be straight or branched and contain from about 2 to about 15 carbon atoms. In one embodiment, an alkynyl group contains from about 2 to about 12 carbon atoms. In another embodiment, an alkynyl group contains from about 2 to about 6 carbon atoms. Non-limiting examples of alkynyl groups include ethynyl, propynyl, 2-butynyl and 3-methylbutynyl. An alkynyl group may be unsubstituted or substituted by one or more substituents which may be the same or different, each substituent being independently selected from the group consisting of halo, alkenyl, alkynyl, aryl, cycloalkyl, cyano, hydroxy, —O-alkyl, —O-aryl, -alkylene-O-alkyl, alkylthio, —NH₂, —NH(alkyl), —N(alkyl)₂, —NH(cycloalkyl), —O—C(O)— alkyl, —O—C(O)-aryl, —O—C(O)-cycloalkyl, —C(O)OH and —C(O)O-alkyl. The term “C₂-C₆ alkynyl” refers to an alkynyl group having from 2 to 6 carbon atoms. Unless otherwise indicated, an alkynyl group is unsubstituted.

The term “alkylene,” as used herein, refers to an alkyl group, as defined above, wherein one of the alkyl group's hydrogen atoms has been replaced with a bond. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 6 carbon atoms. In another embodiment, an alkylene group has from about 3 to about 5 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms. The term “C₃-C₅ alkylene” refers to an alkylene group having from 3 to 5 carbon atoms.

The term “alkenylene,” as used herein, refers to an alkenyl group, as defined above, wherein one of the alkenyl group's hydrogen atoms has been replaced with a bond. Non-limiting examples of alkenylene groups include —CH═CH—, —CH═CHCH₂—, —CH₂CH═CH—, —CH₂CH═CHCH₂—, —CH═CHCH₂CH₂—, —CH₂CH₂CH═CH— and —CH(CH₃)CH═CH—. In one embodiment, an alkenylene group has from 2 to about 6 carbon atoms. In another embodiment, an alkenylene group has from about 3 to about 5 carbon atoms. In another embodiment, an alkenylene group is branched. In another embodiment, an alkenylene group is linear. The term “C₂-C₆ alkylene” refers to an alkenylene group having from 2 to 6 carbon atoms. The term “C₃-C₅ alkenylene” refers to an alkenylene group having from 3 to 5 carbon atoms.

The term “aryl,” as used herein, refers to an aromatic monocyclic or multicyclic ring system comprising from about 6 to about 14 carbon atoms. In one embodiment, an aryl group contains from about 6 to about 10 carbon atoms. An aryl group can be optionally substituted with one or more “ring system substituents” which may be the same or different, and are as defined herein below. In one embodiment, an aryl group can be optionally fused to a cycloalkyl or cycloalkanoyl group. Non-limiting examples of aryl groups include phenyl and naphthyl. In one embodiment, an aryl group is phenyl. Unless otherwise indicated, an aryl group is unsubstituted.

The term “arylene,” as used herein, refers to a bivalent group derived from an aryl group, as defined above, by removal of a hydrogen atom from a ring carbon of an aryl group. An arylene group can be derived from a monocyclic or multicyclic ring system comprising from about 6 to about 14 carbon atoms. In one embodiment, an arylene group contains from about 6 to about 10 carbon atoms. In another embodiment, an arylene group is a naphthylene group. In another embodiment, an arylene group is a phenylene group. An arylene group can be optionally substituted with one or more “ring system substituents” which may be the same or different, and are as defined herein below. An arylene group is divalent and either available bond on an arylene group can connect to either group flanking the arylene group. For example, the group “A-arylene-B,” wherein the arylene group is:

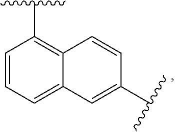 [SMILES: *c1ccc2c(*)cccc2c1]

is understood to represent both:

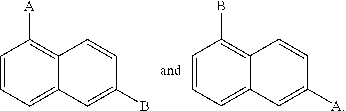

In one embodiment, an arylene group can be optionally fused to a cycloalkyl or cycloalkanoyl group. Non-limiting examples of arylene groups include phenylene and naphthalene. In one embodiment, an arylene group is unsubstituted. In another embodiment, an arylene group is:

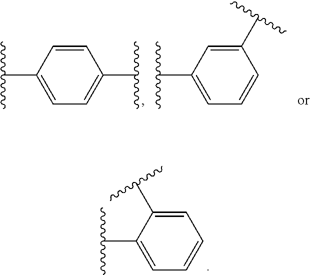

Unless otherwise indicated, an arylene group is unsubstituted.

The term “cycloalkyl,” as used herein, refers to a non-aromatic mono- or multicyclic ring system comprising from about 3 to about 10 ring carbon atoms. In one embodiment, a cycloalkyl contains from about 5 to about 10 ring carbon atoms. In another embodiment, a cycloalkyl contains from about 3 to about 7 ring atoms. In another embodiment, a cycloalkyl contains from about 5 to about 6 ring atoms. The term “cycloalkyl” also encompasses a cycloalkyl group, as defined above, which is fused to an aryl (e.g., benzene) or heteroaryl ring. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl. A cycloalkyl group can be optionally substituted with one or more “ring system substituents” which may be the same or different, and are as defined herein below. In one embodiment, a cycloalkyl group is unsubstituted. The term “3 to 7-membered cycloalkyl” refers to a cycloalkyl group having from 3 to 7 ring carbon atoms. Unless otherwise indicated, a cycloalkyl group is unsubstituted. A ring carbon atom of a cycloalkyl group may be functionalized as a carbonyl group. An illustrative example of such a cycloalkyl group (also referred to herein as a “cycloalkanoyl” group) includes, but is not limited to, cyclobutanoyl:

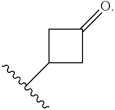 [SMILES: *C1CC(=O)C1]

The term “halo,” as used herein, means —F, —Cl, —Br or —I.

The term “haloalkyl,” as used herein, refers to an alkyl group as defined above, wherein one or more of the alkyl group's hydrogen atoms has been replaced with a halogen. In one embodiment, a haloalkyl group has from 1 to 6 carbon atoms. In another embodiment, a haloalkyl group is substituted with from 1 to 3 F atoms. Non-limiting examples of haloalkyl groups include —CH₂F, —CHF₂, —CF₃, —CH₂Cl and —CCl₃. The term “C₁-C₆ haloalkyl” refers to a haloalkyl group having from 1 to 6 carbon atoms.

The term “hydroxyalkyl,” as used herein, refers to an alkyl group as defined above, wherein one or more of the alkyl group's hydrogen atoms have been replaced with an —OH group. In one embodiment, a hydroxyalkyl group has from 1 to 6 carbon atoms. Non-limiting examples of hydroxyalkyl groups include —CH₂OH, —CH₂CH₂OH, —CH₂CH₂CH₂OH and —CH₂CH(OH)CH₃. The term “C₁-C₆ hydroxyalkyl” refers to a hydroxyalkyl group having from 1 to 6 carbon atoms.

The term “heteroaryl,” as used herein, refers to an aromatic monocyclic or multicyclic ring system comprising about 5 to about 14 ring atoms, wherein from 1 to 4 of the ring atoms is independently O, N or S and the remaining ring atoms are carbon atoms. In one embodiment, a heteroaryl group has 5 to 10 ring atoms. In another embodiment, a heteroaryl group is monocyclic and has 5 or 6 ring atoms. In another embodiment, a heteroaryl group is bicyclic. A heteroaryl group can be optionally substituted by one or more “ring system substituents” which may be the same or different, and are as defined herein below. A heteroaryl group is joined via a ring carbon atom, and any nitrogen atom of a heteroaryl can be optionally oxidized to the corresponding N-oxide. The term “heteroaryl” also encompasses a heteroaryl group, as defined above, which is fused to a benzene ring. Non-limiting examples of heteroaryls include pyridyl, pyrazinyl, furanyl, thienyl, pyrimidinyl, pyridone (including N-substituted pyridones), isoxazolyl, isothiazolyl, oxazolyl, oxadiazolyl, thiazolyl, pyrazolyl, furazanyl, pyrrolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, benzimidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like, and all isomeric forms thereof. The term “heteroaryl” also refers to partially saturated heteroaryl moieties such as, for example, tetrahydroisoquinolyl, tetrahydroquinolyl and the like. In one embodiment, a heteroaryl group is a 5-membered heteroaryl. In another embodiment, a heteroaryl group is a 6-membered monocyclic heteroaryl. In another embodiment, a heteroaryl group comprises a 5- to 6-membered monocyclic heteroaryl group fused to a benzene ring. Unless otherwise indicated, a heteroaryl group is unsubstituted.

The term “heterocycloalkyl,” as used herein, refers to a non-aromatic saturated monocyclic or multicyclic ring system comprising 3 to about 11 ring atoms, wherein from 1 to 4 of the ring atoms are independently O, S, N or Si, and the remainder of the ring atoms are carbon atoms. A heterocycloalkyl group can be joined via a ring carbon, ring silicon atom or ring nitrogen atom. In one embodiment, a heterocycloalkyl group is monocyclic and has from about 3 to about 7 ring atoms. In another embodiment, a heterocycloalkyl group is monocyclic has from about 4 to about 7 ring atoms. In another embodiment, a heterocycloalkyl group is bicyclic and has from about 7 to about 11 ring atoms. In still another embodiment, a heterocycloalkyl group is monocyclic and has 5 or 6 ring atoms. In one embodiment, a heterocycloalkyl group is monocyclic. In another embodiment, a heterocycloalkyl group is bicyclic. There are no adjacent oxygen and/or sulfur atoms present in the ring system. Any —NH group in a heterocycloalkyl ring may exist protected such as, for example, as an —N(BOC), —N(Cbz), —N(Tos) group and the like; such protected heterocycloalkyl groups are considered part of this invention. The term “heterocycloalkyl” also encompasses a heterocycloalkyl group, as defined above, which is fused to an aryl (e.g., benzene) or heteroaryl ring. A heterocycloalkyl group can be optionally substituted by one or more “ring system substituents” which may be the same or different, and are as defined herein below. The nitrogen or sulfur atom of the heterocycloalkyl can be optionally oxidized to the corresponding N-oxide, S-oxide or S,S-dioxide. Non-limiting examples of monocyclic heterocycloalkyl rings include oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, delta-lactam, delta-lactone and the like, and all isomers thereof.

A ring carbon atom of a heterocycloalkyl group may be functionalized as a carbonyl group. An illustrative example of such a heterocycloalkyl group is:

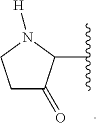 [SMILES: *C1NCCC1=O]

In one embodiment, a heterocycloalkyl group is a 5-membered monocyclic heterocycloalkyl. In another embodiment, a heterocycloalkyl group is a 6-membered monocyclic heterocycloalkyl. The term “3 to 6-membered monocyclic heterocycloalkyl” refers to a monocyclic heterocycloalkyl group having from 3 to 6 ring atoms. The term “4 to 7-membered monocyclic heterocycloalkyl” refers to a monocyclic heterocycloalkyl group having from 4 to 7 ring atoms. The term “7 to 11-membered bicyclic heterocycloalkyl” refers to a bicyclic heterocycloalkyl group having from 7 to 11 ring atoms. Unless otherwise indicated, a heterocycloalkyl group is unsubstituted.

The term “ring system substituent,” as used herein, refers to a substituent group attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, —Si(alkyl)₂, —Si(aryl)₂, —Si(heteroaryl)₂, —Si(alkyl)(aryl), —Si(alkyl)(cycloalkyl), —Si(alkyl)(heteroaryl), cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y₁)(Y₂), -alkylene-N(Y₁)(Y₂), —C(O)N(Y₁)(Y₂) and —S(O)₂N(Y₁)(Y₂), wherein Y₁ and Y₂ can be the same or different and are independently selected from the group consisting of hydrogen, alkyl, aryl, cycloalkyl, and -alkylene-aryl. “Ring system substituent” may also mean a single moiety which simultaneously replaces two available hydrogens on two adjacent carbon atoms (one H on each carbon) on a ring system. Examples of such moiety are methylenedioxy, ethylenedioxy, —C(CH₃)₂— and the like which form moieties such as, for example:

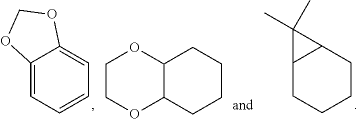

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “in substantially purified form,” as used herein, refers to the physical state of a compound after the compound is isolated from a synthetic process (e.g., from a reaction mixture), a natural source, or a combination thereof. The term “in substantially purified form,” also refers to the physical state of a compound after the compound is obtained from a purification process or processes described herein or well-known to the skilled artisan (e.g., chromatography, recrystallization and the like), in sufficient purity to be characterizable by standard analytical techniques described herein or well-known to the skilled artisan.

It should also be noted that any carbon as well as heteroatom with unsatisfied valences in the text, schemes, examples and tables herein is assumed to have the sufficient number of hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in Organic Synthesis (1991), Wiley, New York.

When any substituent or variable (e.g., alkyl, R¹, R⁷, etc.) occurs more than one time in any constituent or in Formula (I), its definition on each occurrence is independent of its definition at every other occurrence, unless otherwise indicated.

As used herein, the term “composition” is intended to encompass a product comprising the specified ingredients in the specified amounts, as well as any product which results, directly or indirectly, from combination of the specified ingredients in the specified amounts.

Solvates of the compounds of the invention are also contemplated herein.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of solvates include ethanolates, methanolates, and the like. A “hydrate” is a solvate wherein the solvent molecule is water.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTechours., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than room temperature, and cooling the solution at a rate sufficient to form crystals which are then isolated by standard methods. Analytical techniques such as, for example IR spectroscopy, show the presence of the solvent (or water) in the crystals as a solvate (or hydrate).

The Amido-Substituted Pyrimidinone Derivatives can form salts which are also within the scope of this invention. Reference to a Amido-Substituted Pyrimidinone Derivative herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a Amido-Substituted Pyrimidinone Derivative contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. In one embodiment, the salt is a pharmaceutically acceptable (i.e., non-toxic, physiologically acceptable) salt. In another embodiment, the salt is other than a pharmaceutically acceptable salt. Salts of the Compounds of Formula (I) may be formed, for example, by reacting a Amido-Substituted Pyrimidinone Derivative with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Diastereomeric mixtures can be separated into their individual diastereomers on the basis of their physical chemical differences by methods well-known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Sterochemically pure compounds may also be prepared by using chiral starting materials or by employing salt resolution techniques. Also, some of the Amido-Substituted Pyrimidinone Derivatives may be atropisomers (e.g., substituted biaryls) and are considered as part of this invention. Enantiomers can also be directly separated using chiral chromatographic techniques.

It is also possible that the Amido-Substituted Pyrimidinone Derivatives may exist in different tautomeric forms, and all such forms are embraced within the scope of the invention. For example, all keto-enol and imine-enamine forms of the compounds are included in the invention.

All stereoisomers (for example, geometric isomers, optical isomers and the like) of the present compounds (including those of the salts, solvates, hydrates and esters of the compounds), such as those which may exist due to asymmetric carbons on various substituents, including enantiomeric forms (which may exist even in the absence of asymmetric carbons), rotameric forms, atropisomers, and diastereomeric forms, are contemplated within the scope of this invention. If a Amido-Substituted Pyrimidinone Derivative incorporates a double bond or a fused ring, both the cis- and trans-forms, as well as mixtures, are embraced within the scope of the invention.

Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, and the like, is intended to apply equally to the salt, solvate and ester of enantiomers, stereoisomers, rotamers, tautomers, positional isomers or racemates of the inventive compounds.

In the Compounds of Formula (I), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched Compounds of Formula (I) can be prepared without undue experimentation by conventional techniques well known to those skilled in the art or by processes analogous to those described in the Schemes and Examples herein using appropriate isotopically-enriched reagents and/or intermediates. In one embodiment, a Compound of Formula (I) has one or more of its hydrogen atoms replaced with deuterium.

Polymorphic forms of the Amido-Substituted Pyrimidinone Derivatives, and of the salts, solvates, hydrates and esters of the Amido-Substituted Pyrimidinone Derivatives, are intended to be included in the present invention.

The following abbreviations are used below and have the following meanings: AcOH is acetic acid; n-BuLi is n-butyllithium; m-CPBA is 3-chloroperoxybenzoic acid; DABCO is 1,4-diazabicyclo(2,2,2)octane; DEA is diethylamine; DIPEA is N,Ndiisopropylethylamine; DMA is dimethylacetamide; DMF is dimethylformamide; EDCI is 1-ethyl-3-(3′-dimethylaminopropyl)carbodiimide hydrochloride; ESI is electrospray ionization; EtOAc isethyl acetate; EtOH is ethanol; HATU is 2-(1H-7-Azabenzotriazol-1-yl)-1,1,3,3-tetramethyl uronium hexafluorophosphate; HOAt is 1-hydroxy-7-azabenzotriazole; HPLC is high-pressure liquid chromatography; IPA is isopropanol; IPAc is iso-propyl acetate; KOt-Bu is potassium tert-butoxide; LCMS is liquid chromatography-mass spectrometry; LiHMDS is lithium hexamethyldisilylazide; MeCN is acetonitrile; MeOH is methanol; Ms is mesyl or methanesulfonyl; MS is mass spectroscopy; MTBE is methyl tert-butyl ether; NHS is normal human serum; NMR is nuclear magnetic resonance spectroscopy; Piv is pivalate or 2,2-dimethylpropanoyl; Pd/C is palladium on carbon; PyClu is 1-(chloro-1-pyrrolidinylmethylene) pyrrolidinium hexafluorophosphate; SFC is supercritical fluid chromatography; TBAF is n-tetrabutylammonium fluoride; TFA is trifluoroacetic acid; TLC is thin-layer chromatography; Ts is tosyl or 4-toluenesulfonyl; THF is tetrahydrofuran; and Zhan-1b is N-dimethylaminosulfonyl)phenyl]methyleneruthenium(II) dichloride.

### The Compounds of Formula (I)

The present invention provides Amido-Substituted Pyrimidinone Derivatives of Formula (I):

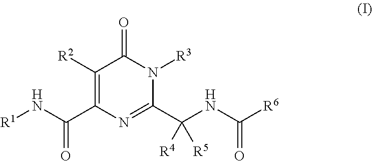

and pharmaceutically acceptable salts thereof, wherein R¹, R², R³, R⁴, R⁵ and R⁶ are defined above for the Compounds of Formula (I).

In one embodiment, R¹ is —(C₁-C₆ alkylene)-(C₆-C₁₀ aryl), which can be optionally substituted with up to four R⁷ groups.

In another embodiment, R¹ is —(C₁-C₆ alkylene)-(5 or 6-membered monocyclic heteroaryl), which can be optionally substituted with up to four R⁷ groups.

In another embodiment, R¹ is —(C₁-C₆ alkylene)-(9 or 10-membered bicyclic heteroaryl), which can be optionally substituted with up to four R⁷ groups.

In still another embodiment, R¹ is —(C₁-C₆ alkylene)-(3 to 7-membered monocyclic heterocycloalkyl), which can be optionally substituted with up to four R⁷ groups.

In another embodiment, R¹ is —(C₁-C₆ alkylene)-(8 to 10-membered bicyclic heterocycloalkyl), which can be optionally substituted with up to four R⁷ groups.

In yet another embodiment, R¹ is —(C₁-C₆ alkylene)-(C₃-C₆ cycloalkyl), which can be optionally substituted with up to four R⁷ groups.

In another embodiment, R¹ is benzyl, and wherein the phenyl moiety of said benzyl group is optionally substituted with one R⁷ group.

In another embodiment, R¹ is 4-fluorobenzyl.

In one embodiment, R² is —O—(C₁-C₆ alkylene)-X—C(O)—Y—R⁹.

In one embodiment, R³ is H.

In another embodiment, R³ is C₁-C₆ alkyl.

In another embodiment, R³ is methyl.

In one embodiment, R⁴ is H.

In another embodiment, R⁴ is C₁-C₆ alkyl.

In another embodiment, R⁴ is methyl.

In one embodiment, R⁵ is H.

In another embodiment, R⁵ is C₁-C₆ alkyl.

In another embodiment, R⁵ is methyl.

In one embodiment, R⁶ is C₆-C₁₀ aryl, which can be optionally substituted with up to four R⁷ groups.

In another embodiment, R⁶ is 5 or 6-membered monocyclic heteroaryl, which can be optionally substituted with up to four R⁷ groups.

In another embodiment, R⁶ is 9 or 10-membered bicyclic heteroaryl, which can be optionally substituted with up to four R⁷ groups.

In yet another embodiment, R⁶ is 3 to 7-membered monocyclic heterocycloalkyl, which can be optionally substituted with up to four R⁷ groups.

In another embodiment, R⁶ is 8 to 10-membered bicyclic heterocycloalkyl, which can be optionally substituted with up to four R⁷ groups.

In still another embodiment, R⁶ is C₃-C₆ cycloalkyl, which can be optionally substituted with up to four R⁷ groups.

In another embodiment, R⁶ is 5-membered monocyclic heteroaryl, which can be optionally substituted with one substituent.

In another embodiment, R⁶ is:

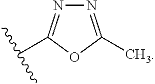 [SMILES: *c1nnc(C)o1]

In one embodiment, R¹ is 4-fluorobenzyl and R⁶ is:

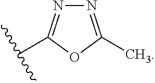 [SMILES: *c1nnc(C)o1]

In one embodiment, R⁴ and R⁵ are each methyl.

In another embodiment, R³, R⁴ and R⁵ are each methyl.

In one embodiment, the compounds of formula (I) have the formula (Ia):

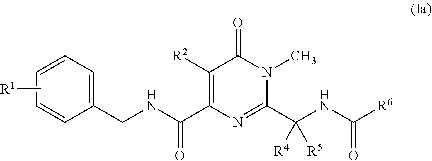

or a pharmaceutically acceptable salt thereof,
wherein:

Y is a bond or O;

R¹ is halo;

R² is —O—(C₁-C₄ alkylene)-O—C(O)—Y—R⁹;

R⁴ is C₁-C₃ alkyl;

R⁵ is C₁-C₃ alkyl;

R⁶ is 5-membered monocyclic heteroaryl, which can be optionally substituted with C₁-C₆ alkyl; and

R⁹ is selected from C₁-C₆ alkyl, C₆-C₁₀ aryl, and C₃-C₆ cycloalkyl, wherein said C₁-C₆ alkyl group can be optionally substituted with phenyl or C₃-C₆ cycloalkyl.

In one embodiment, the compounds of formula (I) have the formula (Ib):

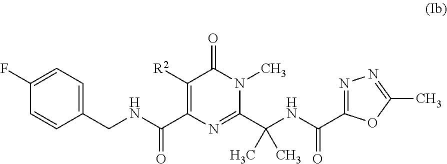

or a pharmaceutically acceptable salt thereof,
wherein:

R² is —O—(CHR⁷)—O—C(O)—Y—R⁹;

Y is O or a bond;

R⁷ is H, methyl, ethyl or isopropyl;

R⁹ is C₁-C₄ alkyl, —(CH₂)ₙ—C₃-C₆ cycloalkyl or —(CH₂)ₙ-phenyl; and

n is 0 or 1.

In one embodiment, for the compounds of Formula (I), (II) or (III), R² is —O—(C₁-C₆ alkylene)-O—C(O)OR⁹.

In another embodiment, for the compounds of Formula (I), (II) or (III), R² is —O—(C₁-C₆ alkylene)-O—C(O)R⁹.

In another embodiment, for the compounds of Formula (I), (II) or (III), R² is selected from:

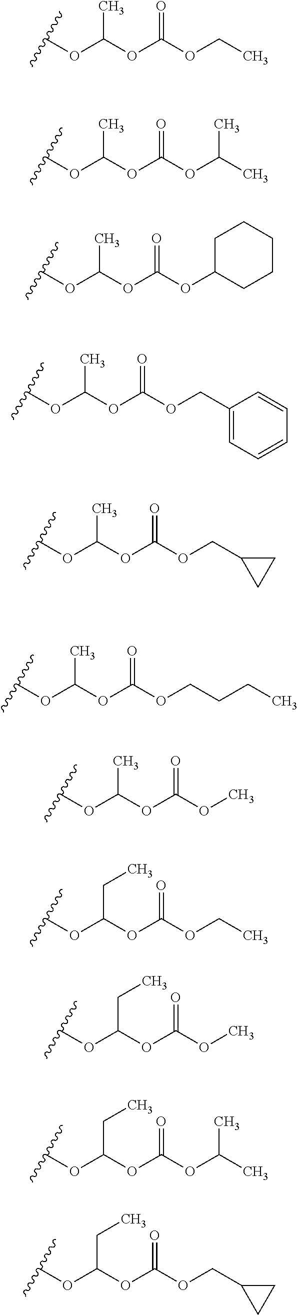

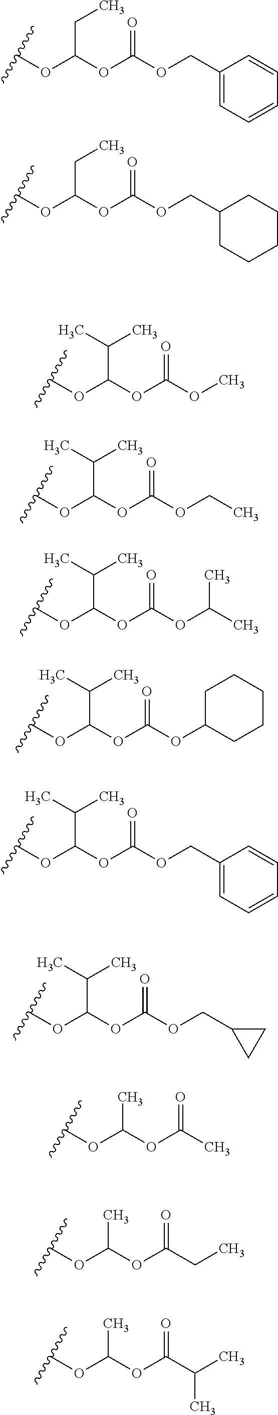

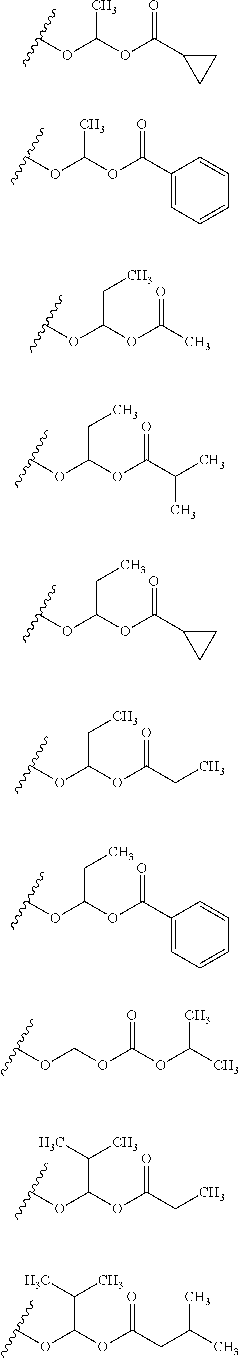

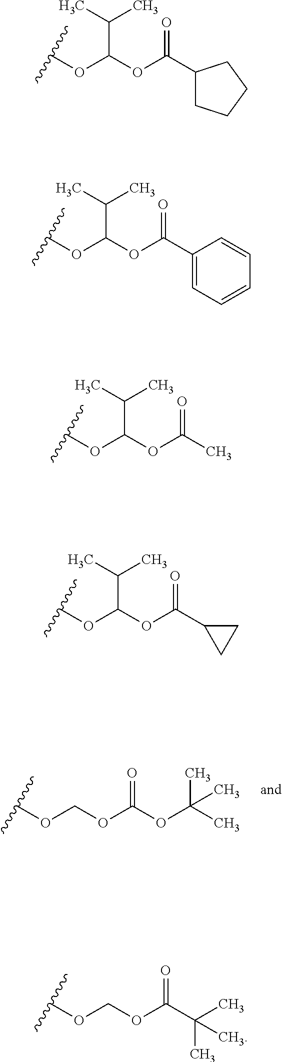

In still another embodiment, for the compounds of Formula (I), (II) or (III), R² is selected from:

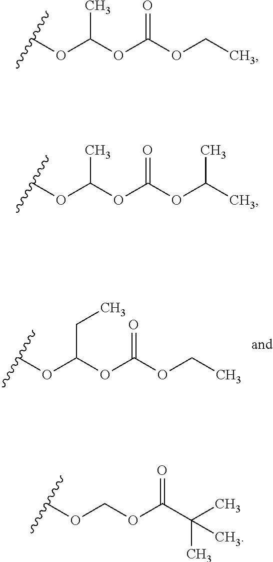

In one embodiment, for the Compounds of Formula (I), variables R¹, R², R³, R⁴, R⁵ and R⁶ are selected independently of each other.

In another embodiment, the Compounds of Formula (I) are in substantially purified form.

Other embodiments of the present invention include the following:

(a) A pharmaceutical composition comprising an effective amount of a Compound of Formula (I) or a pharmaceutically acceptable salt thereof, and a pharmaceutically acceptable carrier.

(b) The pharmaceutical composition of (a), further comprising a second therapeutic agent selected from the group consisting of HIV antiviral agents, immunomodulators, and anti-infective agents.

(c) The pharmaceutical composition of (b), wherein the HIV antiviral agent is an antiviral selected from the group consisting of HIV protease inhibitors, HIV integrase inhibitors, CCR5 co-receptor antagonists, nucleoside reverse transcriptase inhibitors and non-nucleoside reverse-transcriptase inhibitors.

(d) A pharmaceutical combination that is (i) a Compound of Formula (I) and (ii) a second therapeutic agent selected from the group consisting of HIV antiviral agents, immunomodulators, and anti-infective agents; wherein the Compound of Formula (I) and the second therapeutic agent are each employed in an amount that renders the combination effective for inhibiting HIV replication, or for treating HIV infection and/or reducing the likelihood or severity of symptoms of HIV infection.

(e) The combination of (d), wherein the HIV antiviral agent is an antiviral selected from the group consisting of HIV protease inhibitors, HIV integrase inhibitors, CCR5 co-receptor antagonists, nucleoside reverse transcriptase inhibitors and non-nucleoside reverse-transcriptase inhibitors.

(f) A method of inhibiting HIV replication in a subject in need thereof which comprises administering to the subject an effective amount of a Compound of Formula (I).

(g) A method of treating HIV infection and/or reducing the likelihood or severity of symptoms of HIV infection in a subject in need thereof which comprises administering to the subject an effective amount of a Compound of Formula (I).

(h) The method of (g), wherein the Compound of Formula (I) is administered in combination with an effective amount of at least one second therapeutic agent selected from the group consisting of HIV antiviral agents, immunomodulators, and anti-infective agents.

(i) The method of (h), wherein the HIV antiviral agent is an antiviral selected from the group consisting of HIV protease inhibitors, HIV integrase inhibitors, CCR5 co-receptor antagonists, nucleoside reverse transcriptase inhibitors and non-nucleoside reverse-transcriptase inhibitors.

(j) A method of inhibiting HIV replication in a subject in need thereof which comprises administering to the subject the pharmaceutical composition of (a), (b) or (c) or the combination of (d) or (e).

(k) A method of treating HIV infection and/or reducing the likelihood or severity of symptoms of HIV infection in a subject in need thereof which comprises administering to the subject the pharmaceutical composition of (a), (b) or (c) or the combination of (d) or (e).

The present invention also includes a compound of the present invention for use (i) in, (ii) as a medicament for, or (iii) in the preparation of a medicament for: (a) medicine, (b) inhibiting HIV replication or (c) treating HIV infection and/or reducing the likelihood or severity of symptoms of HIV infection. In these uses, the compounds of the present invention can optionally be employed in combination with one or more second therapeutic agents selected from HIV antiviral agents, anti-infective agents, and immunomodulators.

Additional embodiments of the invention include the pharmaceutical compositions, combinations and methods set forth in (a)-(k) above and the uses set forth in the preceding paragraph, wherein the compound of the present invention employed therein is a compound of one of the embodiments, aspects, classes, sub-classes, or features of the compounds described above. In all of these embodiments, the compound may optionally be used in the form of a pharmaceutically acceptable salt or hydrate as appropriate. It is understood that references to compounds would include the compound in its present form as well as in different forms, such as polymorphs, solvates and hydrates, as applicable.

It is further to be understood that the embodiments of compositions and methods provided as (a) through (k) above are understood to include all embodiments of the compounds, including such embodiments as result from combinations of embodiments.

The Compounds of Formula (I) may be referred to herein by chemical structure and/or by chemical name. In the instance that both the structure and the name of a Compound of Formula (I) are provided and a discrepancy is found to exist between the chemical structure and the corresponding chemical name, it is understood that the chemical structure will predominate.

Non-limiting examples of the Compounds of Formula (I) include compounds 1-38 as set forth below, and pharmaceutically acceptable salts thereof

### Methods for Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and generalized in Schemes A and B below. Alternative synthetic pathways and analogous structures will be apparent to those skilled in the art of organic synthesis.

Scheme 1 describes a method useful for making compounds of formula C, which correspond to the Compounds of Formula (I) wherein R² is —O—(C₁-C₆ alkylene)-O—C(O)—O—R⁹.

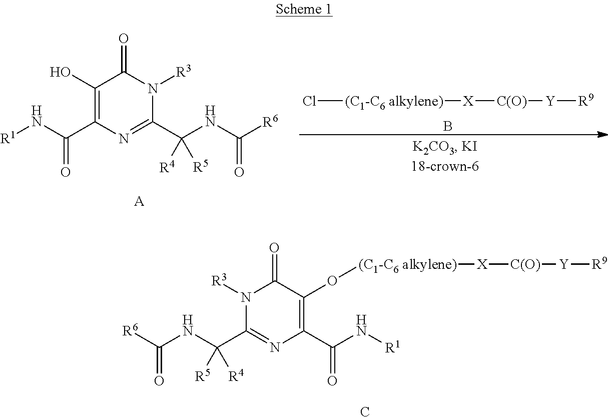

Wherein R¹, R², R³, R⁴, R⁵, R⁶ and R⁹ are defined above for the Compounds of Formula (I).

A compound of formula A (which can be prepared, for example, using the methods described in Organic Process Research & Development, 2011, 15, 73-83) can be reacted with a chlorocarbonate of formula B (which can be prepared using methods well-known to those skilled in the art of organic synthesis, for example, using the methods described in WO 2010/011814 A1) to provide compounds of formula C, which correspond to the Compounds of Formula (I) wherein R² is —O—(C₁-C₆ alkylene)-O—C(O)—O—R⁹.

Scheme 2 describes a method useful for making compounds of formula E, which correspond to the Compounds of Formula (I) wherein R² is —O—(C₁-C₆ alkylene)-O—C(O)—R⁹.

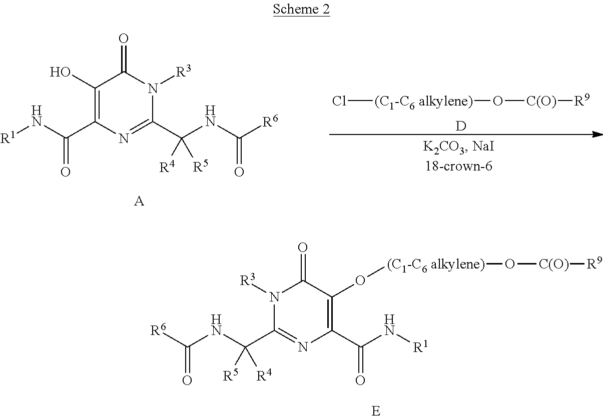

Wherein R¹, R², R³, R⁴, R⁵, R⁶ and R⁹ are defined above for the Compounds of Formula (I).

A compound of formula A (which can be prepared, for example, using the methods described in Organic Process Research & Development, 2011, 15, 73-83) can be reacted with a chlorocarbonate of formula D (which can be prepared using methods well-known to those skilled in the art of organic synthesis) to provide compounds of formula E, which correspond to the Compounds of Formula (I) wherein R² is —O—(C₁-C₆ alkylene)-O—C(O)—R⁹.

One skilled in the art of organic synthesis will recognize that the synthesis of compounds with multiple reactive functional groups, such as —OH and NH₂, may require protection of certain functional groups (i.e., derivatization for the purpose of chemical compatibility with a particular reaction condition). Suitable protecting groups for the various functional groups of these compounds and methods for their installation and removal are well-known in the art of organic chemistry. A summary of many of these methods can be found in Greene & Wuts, Protecting Groups in Organic Synthesis, John Wiley & Sons, 3^{rd} edition (1999).

One skilled in the art of organic synthesis will also recognize that one route for the synthesis of the Compounds of Formula (I) may be more desirable depending on the choice of appendage substituents. Additionally, one skilled in the relevant art will recognize that in some cases the order of reactions may differ from that presented herein to avoid functional group incompatibilities and thus adjust the synthetic route accordingly.

Compounds of formula C and E may be further elaborated using methods that would be well-known to those skilled in the art of organic synthesis or, for example, the methods described in the Examples below, to make the full scope of the Compounds of Formula (I).

The starting materials used and the intermediates prepared using the methods set forth in Schemes A and B may be isolated and purified if desired using conventional techniques, including but not limited to filtration, distillation, crystallization, chromatography and alike. Such materials can be characterized using conventional means, including physical constants and spectral data.

### EXAMPLES

### General Methods

The compounds described herein can be prepared according to the procedures of the following schemes and examples, using appropriate materials and are further exemplified by the following specific examples. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds. All temperatures are degrees Celsius unless otherwise noted. Mass spectra (MS) were measured by electrospray ion-mass spectroscopy (ESI). ¹H NMR spectra were recorded at 400-500 MHz. Compounds described herein were synthesized as a racemic mixture unless otherwise stated in the experimental procedures.

### Example 1

### Preparation of Compound 1

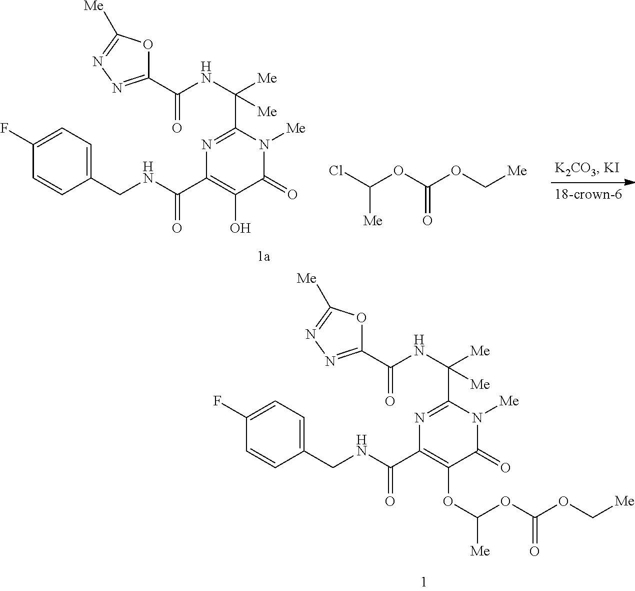 [SMILES: Cc1nnc(C(=O)NC(C)(C)c2nc(C(=O)NCc3ccc(F)cc3)c(O)c(=O)n2C)o1 ; CCOC(=O)OC(C)Cl ; CCOC(=O)OC(C)Oc1c(C(=O)NCc2ccc(F)cc2)nc(C(C)(C)NC(=O)c2nnc(C)o2)n(C)c1=O]

A mixture of compound 1a (31.0 g, 67 mmol, made using the method described in U.S. Pat. No. 7,169,780), 1-chloroethyl ethyl carbonate (14.7 mL, 96 mmol), potassium carbonate (13.3 g, 96 mmol), potassium iodide (13.3 g, 96 mmol), and 18-crown-6 (0.85 g, 3.2 mmol) in DMF (250 mL) was heated at 60° C. and allowed to stir at this temperature for about 4 hours. The resultant reaction mixture was cooled to room temperature and diluted with ethyl acetate. The solution was washed successively with water, and brine, then dried over anhydrous sodium sulfate, filtered, and concentrated in vacuo. The resulting residue was purified using flash chromatography on silica gel (20% ethanol/ethyl acetate) and the solid product obtained was further purified via recrystallization from a mixture of ethyl acetate:hexanes (1:2) to provide compound 1 as a white solid. ¹H NMR (500 MHz, DMSO-d6): δ 9.92 (s, 1H), 8.75 (t, 1H), 7.38 (dd, 2H), 7.16 (t, 2H), 6.31 (q, 1H), 4.43 (s, 2H), 4.00 (q, 2H), 3.49 (s, 3H), 2.57 (s, 3H), 1.72 (d, 6H), 1.54 (d, 3H), 1.13 (t, 3H). ESI+MS: (M+H) 561.1; 471.1, 445.1, 387.1.

The following compounds of the present invention were made using the method described in Example 1 and substituting the appropriate reactants and reagents.

| Compound | | Spectroscopic |
|---|---|---|
| No. | Structure | Data |
| 2 | 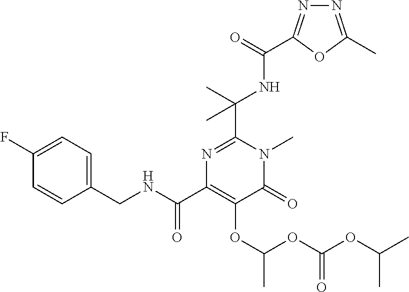 [SMILES: Cc1nnc(C(=O)NC(C)(C)c2nc(C(=O)NCc3ccc(F)cc3)c(OC(C)OC(=O)OC(C)C)c(=O)n2C)o1] | (400 MHz, CDCl₃) δ 8.21 (s, 1H), 7.80-7.83 (m, 1H), 7.35-7.39 (m, 2H), 6.99- 7.04 (m, 2H), 6.47-6.51 (m, 1H), 4.69-4.75 (m, 1H), 4.58 (d, J = 6.0 Hz, 2H), 3.63 (s, 3H), 2.63 (s, 3H), 1.89 (d, J = 2.8 Hz, 6H), 1.77 (d, J = 5.2 Hz, 3H), 1.21-1.25 (m, 6H). LCMS 575.2 (M + 1)⁺ |
| 3 | 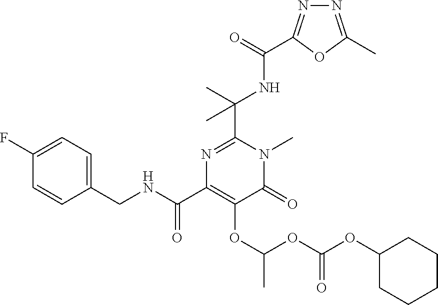 [SMILES: Cc1nnc(C(=O)NC(C)(C)c2nc(C(=O)NCc3ccc(F)cc3)c(OC(C)OC(=O)OC3CCCCC3)c(=O)n2C)o1] | (400 MHz, CDCl₃) δ 8.21 (s, 1H), 7.79-7.82 (m, 1H), 7.35-7.39 (m, 2H), 7.00- 7.04 (m, 2H), 6.49-6.53 (m, 1H), 4.58 (d, J = 6.0 Hz, 2H), 4.45-4.49 (m, 1H), 3.63 (s, 3H), 2.63 (s, 3H), 1.88 (d, J = 3.6 Hz, 6H), 1.81-1.82 (m, 2H), 1.77 (d, J = 5.2 Hz, 3H), 1.70-1.72 (m, 2H), 1.41-1.50 (m, 3H), 1.20-1.38 (m, 3H). LCMS 615.2 (M + 1)⁺ |
| 4 | 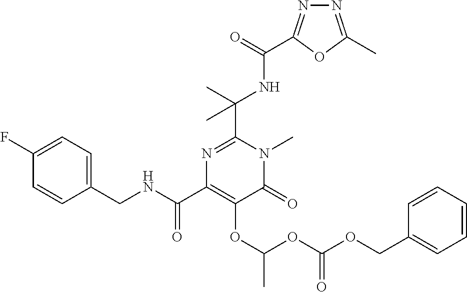 [SMILES: Cc1nnc(C(=O)NC(C)(C)c2nc(C(=O)NCc3ccc(F)cc3)c(OC(C)OC(=O)OCc3ccccc3)c(=O)n2C)o1] | (400 MHz, CDCl₃) δ 8.18 (s, 1H), 7.73-7.76 (m, 1H), 7.31-7.36 (m, 7H), 6.95- 6.99 (m, 2H), 6.50-6.54 (m, 1H), 5.05 (d, J = 2.0 Hz, 2H), 4.55 (d, J = 6.0 Hz, 2H), 3.60 (s, 3H), 2.63 (s, 3H), 1.88 (s, 6H), 1.77 (d, J = 5.2 Hz, 3H). LCMS 623.2 (M + 1)⁺. |
| 5 | 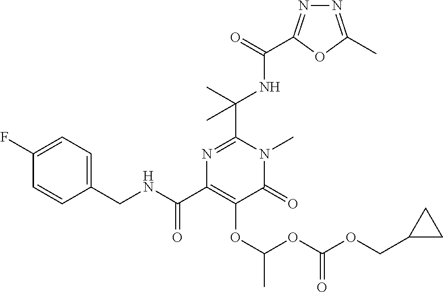 [SMILES: Cc1nnc(C(=O)NC(C)(C)c2nc(C(=O)NCc3ccc(F)cc3)c(OC(C)OC(=O)OCC3CC3)c(=O)n2C)o1] | (400 MHz, CDCl₃) δ 8.19 (s, 1H), 7.78-7.81 (m, 1H), 7.35-7.39 (m, 2H), 7.00- 7.04 (m, 2H), 6.49-6.53 (m, 1H), 4.58-4.59 (m, 2H), 3.83~3.86 (m, 2H), 3.63 (s, 3H), 2.63 (s, 3H), 1.89 (d, J = 2.4 Hz, 6H), 1.74 (d, J = 5.6 Hz, 3H), 1.07-1.14 (m, 1H), 0.52-0.56 (m, 2H), 0.25-0.27 (m, 2H). LCMS 587.3 (M + 1)⁺ |
| 6 | 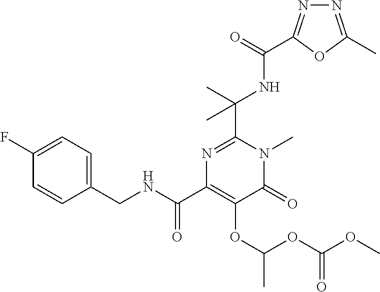 [SMILES: COC(=O)OC(C)Oc1c(C(=O)NCc2ccc(F)cc2)nc(C(C)(C)NC(=O)c2nnc(C)o2)n(C)c1=O] | (400 MHz, CDCl₃) δ 8.20 (s, 1H), 7.79-8.01 (m, 1H), 7.35-7.38 (m, 2H), 7.00- 7.04 (m, 2H), 6.48-6.49 (m, 1H), 4.59 (d, J = 5.6 Hz, 2H), 3.67 (d, J = 14.8 Hz, 6H), 2.63 (s, 3H), 1.88 (s, 6H), 1.76 (d, J = 5.2 Hz, 3H). LCMS 547.2 (M + 1) |
| 7 | 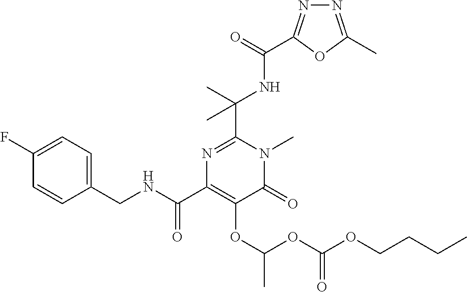 [SMILES: CCCCOC(=O)OC(C)Oc1c(C(=O)NCc2ccc(F)cc2)nc(C(C)(C)NC(=O)c2nnc(C)o2)n(C)c1=O] | (400 MHz, CDCl₃) δ 8.27 (s, 1H), 7.79-7.82 (m, 1H), 7.34-7.37 (m, 2H), 6.98- 7.04 (m, 2H), 6.46-6.50 (m, 1H), 4.58 (d, J = 6.0 Hz, 2H), 4.00-4.03 (m, 2H), 3.63 (s, 3H), 2.61 (s, 3H), 1.88 (d, J = 2.4 Hz, 6H), 1.75 (d, J = 5.2 Hz, 3H), 1.55-1.76 (m, 2H), 1.30- 1.39 (m, 2H), 0.87-0.90 (m, 3H). LCMS 589.2 (M + 1)⁺ |
| 8 | 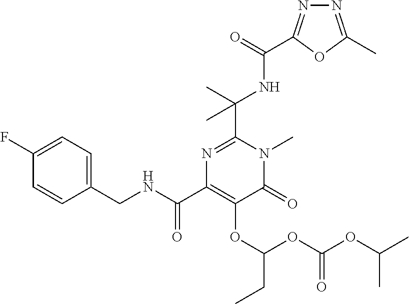 [SMILES: CCC(OC(=O)OC(C)C)Oc1c(C(=O)NCc2ccc(F)cc2)nc(C(C)(C)NC(=O)c2nnc(C)o2)n(C)c1=O] | (400 MHz, CDCl₃) δ 8.26 (s, 1H), 7.80-7.83 (m, 1H), 7.35-7.39 (m, 2H), 6.99- 7.04 (m, 2H), 6.41-6.44 (m, 1H), 4.69-4.76 (m, 1H), 4.58 (d, J = 6.0 Hz, 2H), 3.63 (s, 3H), 2.63 (s, 3H), 2.01-2.19 (m, 2H), 1.88 (d, J = 4.8 Hz, 6H), 1.18-1.24 (m, 6H), 1.08 (t, J = 7.2 Hz, 3H). LCMS 589.2 (M + 1)⁺ |
| 9 | 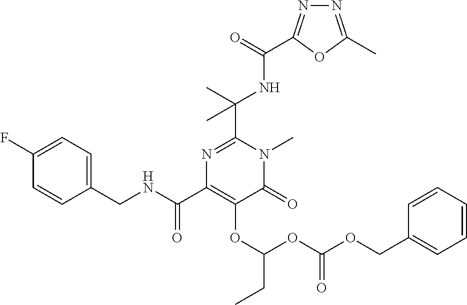 [SMILES: CCC(OC(=O)OCc1ccccc1)Oc1c(C(=O)NCc2ccc(F)cc2)nc(C(C)(C)NC(=O)c2nnc(C)o2)n(C)c1=O] | (400 MHz, CDCl₃) δ 8.23 (s, 1H), 7.73-7.76 (m, 1H), 7.31-7.36 (m, 7H), 6.95- 6.99 (m, 2H), 6.50-6.54 (m, 1H), 5.05 (d, J = 2.0 Hz, 2H), 4.53 (d, J = 6.0 Hz, 2H), 3.61 (s, 3H), 2.62 (s, 3H), 2.01-2.19 (m, 2H), 1.88 (d, J = 2.4 Hz, 6H), 1.06 (d, J = 7.2 Hz, 3H). LCMS 637.1 (M + 1)⁺ |
| 10 | 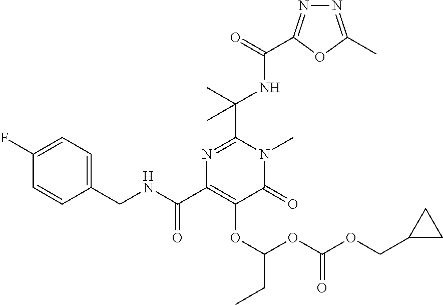 [SMILES: CCC(OC(=O)OCC1CC1)Oc1c(C(=O)NCc2ccc(F)cc2)nc(C(C)(C)NC(=O)c2nnc(C)o2)n(C)c1=O] | (400 MHz, CDCl₃) δ 8.22 (s, 1H), 7.78-7.81 (m, 1H), 7.35-7.39 (m, 2H), 7.00- 7.04 (m, 2H), 6.49-6.53 (m, 1H), 4.58-4.59 (d, J = 6.0 Hz, 2H), 3.83-3.86 (m, 2H), 3.63 (s, 3H), 2.63 (s, 3H), 2.01-2.19 (m, 2H), 1.88 (d, J = 4.0 Hz, 6H), 1.07-1.13 (m, 4H), 0.52-0.57 (m, 2H), 0.25-0.27 (m, 2H). LCMS 600.2 (M + 1)⁺ |
| 11 | 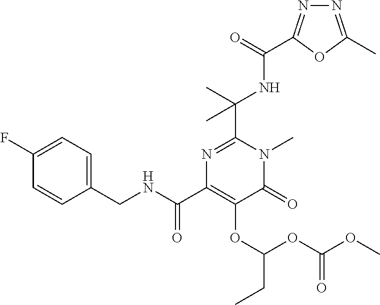 [SMILES: CCC(OC(=O)OC)Oc1c(C(=O)NCc2ccc(F)cc2)nc(C(C)(C)NC(=O)c2nnc(C)o2)n(C)c1=O] | (400 MHz, CDCl₃) δ 8.22 (s, 1H), 7.79-8.01 (m, 1H), 7.35-7.38 (m, 2H), 7.00- 7.04 (m, 2H), 6.48-6.49 (m, 1H), 4.59 (d, J = 6.0 Hz, 2H), 3.65 (d, J = 14.8 Hz, 6H), 2.63 (s, 3H), 2.01-2.19 (m, 2H), 1.89 (d, J = 3.2 Hz, 6H), 1.06 (d, J = 7.2 Hz, 3H). LCMS 561.2 (M + 1)⁺ |
| 12 | 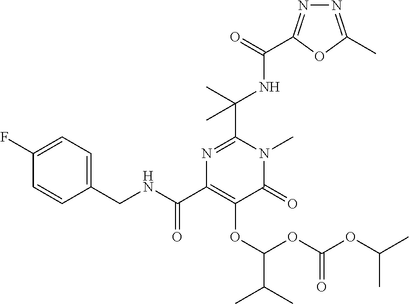 [SMILES: Cc1nnc(C(=O)NC(C)(C)c2nc(C(=O)NCc3ccc(F)cc3)c(OC(OC(=O)OC(C)C)C(C)C)c(=O)n2C)o1] | (400 MHz, CDCl₃) δ 8.21 (brs, 1H), 7.74 (t, J = 6.0 Hz, 1H), 7.35-7.38 (m, 2H), 7.02 (t, J = 8.8 Hz, 2H), 6.44 (d, J = 4.4 Hz, 1H), 4.71-4.74 (m, 1H), 4.57- 4.60 (m, 2H), 3.64 (s, 3H), 2.63 (s, 3H), 2.28-2.31 (m, 1H), 1.82 (s, 3H), 1.81 (s, 3H), 1.24 (d, J = 4.0 Hz, 3H), 1.16 (d, J = 6.0 Hz, 3H), 1.03-1.07 (m, 6H). LCMS 603.3 (M + 1)⁺ |
| 13 | 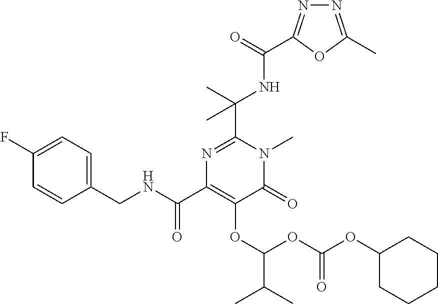 [SMILES: Cc1nnc(C(=O)NC(C)(C)c2nc(C(=O)NCc3ccc(F)cc3)c(OC(OC(=O)OC3CCCCC3)C(C)C)c(=O)n2C)o1] | (400 MHz, CDCl₃) δ 8.24 (brs, 1H), 7.74 (t, J = 5.6 Hz, 1H), 7.35-7.39 (m, 2H), 7.03 (t, J = 8.4 Hz, 2H), 6.46 (d, J = 4.4 Hz, 1H), 4.54-4.64 (m, 2H), 4.42- 4.38 (m, 1H), 3.64 (s, 3H), 2.63 (s, 3H), 2.27-2.31 (m, 1H), 1.90 (s, 3H), 1.88 (s, 3H), 1.65-1.74 (m, 4H), 1.24-1.31 (m, 6H), 1.07 (d, J = 6.8 Hz, 3H), 1.05 (d, J = 6.8 Hz, 3H). LCMS 665.3 (M + 23)⁺ |
| 14 | 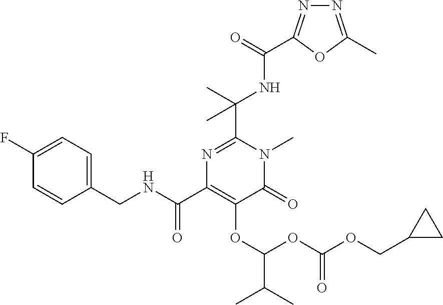 [SMILES: Cc1nnc(C(=O)NC(C)(C)c2nc(C(=O)NCc3ccc(F)cc3)c(OC(OC(=O)OCC3CC3)C(C)C)c(=O)n2C)o1] | (400 MHz, CDCl₃) δ 8.21 (brs, 1H), 7.81 (t, J = 5.2 Hz, 1H), 7.35-7.38 (m, 2H), 7.04 (t, J = 8.8 Hz, 2H), 6.47 (d, J = 4.4 Hz, 1H), 4.58-4.62 (m, 2H), 3.87 (m, 2H), 3.67 (s, 3H), 2.63 (s, 3H), 2.25-2.30 (m, 2H), 1.88 (s, 3H), 1.87 (s, 3H), 1.06 (d, J = 6.8 Hz, 3H), 1.03 (d, J = 6.8 Hz, 3H), 0.51-0.55 (m, 2H), 0.20- 0.25 (m, 2H). LCMS 637.2 (M + 23)⁺ |
| 15 | 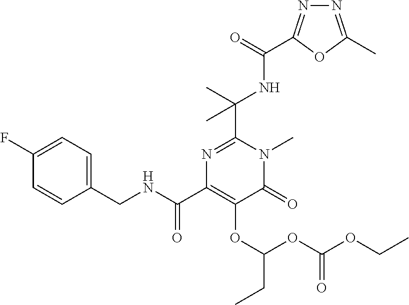 [SMILES: CCOC(=O)OC(CC)Oc1c(C(=O)NCc2ccc(F)cc2)nc(C(C)(C)NC(=O)c2nnc(C)o2)n(C)c1=O] | (400 MHz, CDCl₃) δ 8.26 (s, 1H), 7.79-7.82 (m, 1H), 7.34-7.37 (m, 2H), 6.98- 7.04 (m, 2H), 6.40-6.43 (m, 1H), 4.58 (d, J = 6.0 Hz, 2H), 4.02-4.10 (m, 2H), 3.63 (s, 3H), 2.62 (s, 3H), 2.01-2.19 (m, 2H), 1.88 (d, J = 2.4 Hz, 6H), 1.23 (t, J = 7.2 Hz, 3H), 1.06 (d, J = 7.2 Hz, 3H). LCMS 575.0 (M + 1)⁺ |
| 16 | 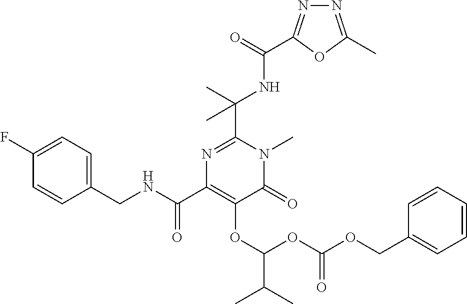 [SMILES: Cc1nnc(C(=O)NC(C)(C)c2nc(C(=O)NCc3ccc(F)cc3)c(OC(OC(=O)OCc3ccccc3)C(C)C)c(=O)n2C)o1] | (400 MHz, CDCl₃) δ 8.17 (brs, 1H), 7.58 (brs, 1H), 7.30-7.33 (m, 7H), 6.95 (t, J = 8.8 Hz, 2H), 6.46 (d, J = 4.4 Hz, 1H), 5.01-5.10 (m, 2H), 4.51-4.55 (m, 2H), 3.63 (s, 3H), 2.64 (s, 3H), 2.30-2.36 (m, 1H), 1.89 (s, 3H), 1.88 (s, 3H), 1.08 (d, J = 6.8 Hz, 3H), 1.05 (d, J = 6.8 Hz, 3H). LCMS 673.1 (M + 23)⁺ |
| 17 | 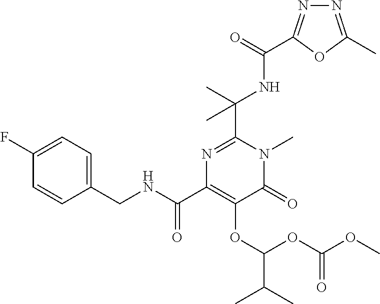 [SMILES: COC(=O)OC(Oc1c(C(=O)NCc2ccc(F)cc2)nc(C(C)(C)NC(=O)c2nnc(C)o2)n(C)c1=O)C(C)C] | (400 MHz, CDCl₃) δ 8.21 (brs, 1H), 7.76 (s, 1H), 7.34-7.38 (m, 2H), 7.03 (t, J = 8.8 Hz, 2H), 6.43 (d, J = 4.0 Hz, 1H), 4.59-4.62 (m, 2H), 3.68 (s, 3H), 3.65 (s, 3H), 2.63 (s, 3H), 2.25-2.30 (m, 1H), 1.89 (s, 3H), 1.88 (s, 3H), 1.05 (d, J = 6.8 Hz, 3H), 1.03 (d, J = 6.8 Hz, 3H) LCMS 597.2 (M + 23)⁺ |
| 18 | 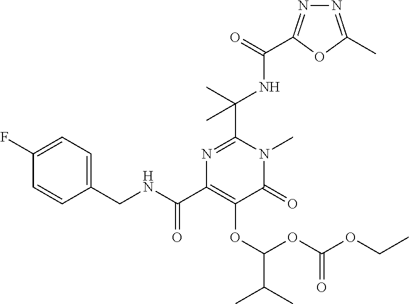 [SMILES: CCOC(=O)OC(Oc1c(C(=O)NCc2ccc(F)cc2)nc(C(C)(C)NC(=O)c2nnc(C)o2)n(C)c1=O)C(C)C] | (400 MHz, CDCl₃) δ 8.20 (brs, 1H), 7.69 (brs, 1H), 7.35-7.39 (m, 2H), 7.02 (t, J = 6.8 Hz, 2H), 6.43 (d, J = 4.4 Hz, 1H), 4.59-4.61 (m, 2H), 4.05-4.10 (q, J = 7.2 Hz, 2H), 3.65 (s, 3H), 2.63 (s, 3H), 2.30-2.35 (m, 1H), 1.91 (s, 3H), 1.89 (s, 3H), 1.23 (t, J = 7.2 Hz, 3H), 1.08 (d, J = 6.8 Hz, 3H), 1.05 (d, J = 6.8 Hz, 3H). LCMS 611.1 (M + 23)⁺ |
| 19 | 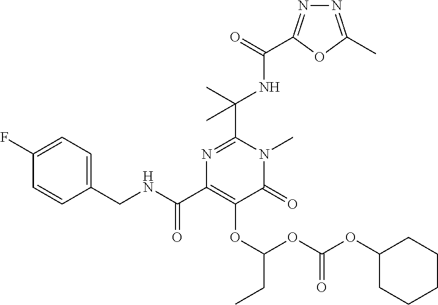 [SMILES: CCC(OC(=O)OC1CCCCC1)Oc1c(C(=O)NCc2ccc(F)cc2)nc(C(C)(C)NC(=O)c2nnc(C)o2)n(C)c1=O] | (400 MHz, CDCl₃) δ 8.26 (s, 1H), 7.74-7.77 (m, 1H), 7.35-7.39 (m, 2H), 7.00- 7.04 (m, 2H), 6.49-6.53 (m, 1H), 4.58 (d, J = 6.0 Hz, 2H), 4.43-4.48 (m, 1H), 3.62 (s, 3H), 2.63 (s, 3H), 2.01-2.19 (m, 2H), 1.77- 1.88 (m, 10H), 1.13-1.49 (m, 6H), 1.08 (t, J = 7.2 Hz, 3H). LCMS 651.2 (M + 23)⁺ |
| 20 | 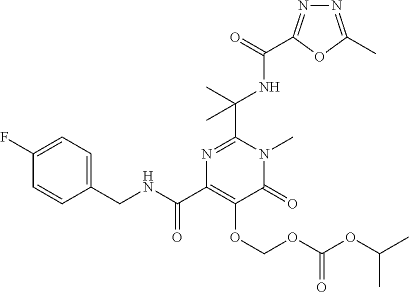 [SMILES: Cc1nnc(C(=O)NC(C)(C)c2nc(C(=O)NCc3ccc(F)cc3)c(OCOC(=O)OC(C)C)c(=O)n2C)o1] | (400 MHz, CDCl₃) δ 9.90 (s, 1H), 8.71~8.74 (m, 1H), 7.37~7.40 (m, 2H), 7.10~7.15 (m, 2H), 6.30~6.31 (m, 1H), 4.69- 4.75 (m, 1H), 4.33~4.47 (m, 2H), 3.46 (s, 3H), 2.55 (s, 3H), 2.36~2.37 (m, 1H), 1.67 (s, 3H), 0.93~0.97 (m, 3H), 1.21-1.25 (m, 6H). LCMS 561.2 (M + 1)⁺ |
| 21 | 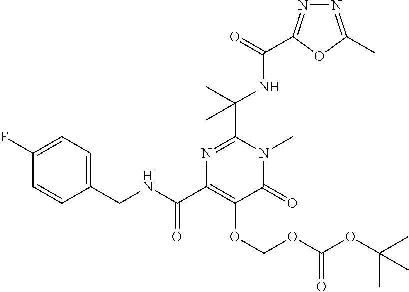 [SMILES: Cc1nnc(C(=O)NC(C)(C)c2nc(C(=O)NCc3ccc(F)cc3)c(OCOC(=O)OC(C)(C)C)c(=O)n2C)o1] | (400 MHz, CDCl₃) δ 9.90 (s, 1H), 8.71~8.74 (m, 1H), 7.37~7.40 (m, 2H), 7.10~7.15 (m, 2H), 6.30~6.31 (m, 1H), 4.33~4.47 (m, 2H), 3.46 (s, 3H), 2.55 (s, 3H), 2.36~2.37 (m, 1H), 1.67 (s, 3H), 0.93~0.97 (m, 3H), 0.91~0.92 (m, 9H). LCMS 575.2 (M + 1)⁺ |

### Example 2

### Preparation of Compound 22

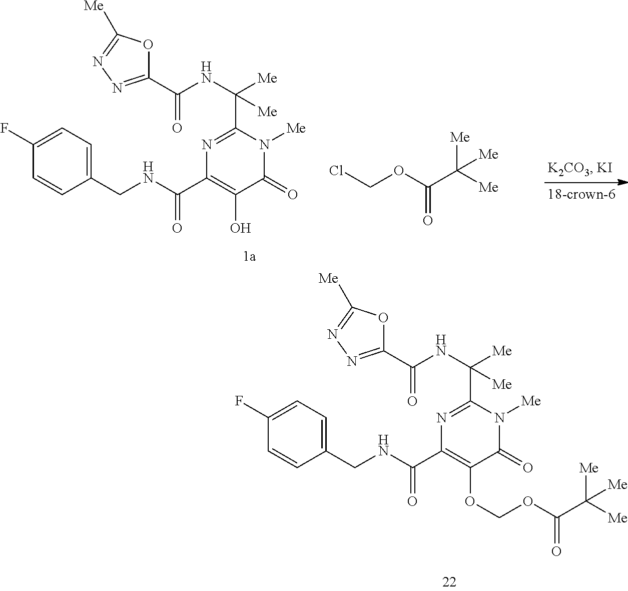 [SMILES: Cc1nnc(C(=O)NC(C)(C)c2nc(C(=O)NCc3ccc(F)cc3)c(O)c(=O)n2C)o1 ; CC(C)(C)C(=O)OCCl ; Cc1nnc(C(=O)NC(C)(C)c2nc(C(=O)NCc3ccc(F)cc3)c(OCOC(=O)C(C)(C)C)c(=O)n2C)o1]

A mixture of compound 1a (31.1 g, 67 mmol, made using the method described in U.S. Pat. No. 7,169,780), chloromethylpivalate (15.8 g, 105 mmol), potassium carbonate (14.5 g, 105 mmol), sodium iodide (21 g, 140 mmol), and 18-crown-6 (0.93 g, 3.5 m mol.) in DMF (175 mL) was heated at 50° C. for 4 hr. The resultant reaction mixture was cooled to room temperature and diluted with ethyl acetate. The organic solution was washed successively with water, and brine. The organic phase was dried over anhydrous sodium sulfate, filtered, and concentrated under vacuum. The residue was subjected to flash chromatography on silica gel eluting with 90% ethyl acetate in hexane. Collection and concentration of appropriate fractions afforded the desired product as a white solid. ¹H NMR (500 MHz, CDCl3): δ 8.16 (s, 1H), 7.89 (t, J=6 Hz, 1H), 7.38 (dd, J=8 Hz, J=5.6 Hz, 2H), 7.02 (t, J=8 Hz, 2H), 5.92 (s, 2H), 4.57 (d, J=6.0 Hz, 2H), 3.65 (s, 3H), 2.65 (s, 3H), 1.90 (s, 6H), 1.18 (s, 9H). LCMS anal. calcd. for C₂₆H₃₁FN₆O₇: 558.2; Found: 559.1 (M+1)⁺

The following compounds of the present invention were made using the method described in Example 2 and substituting the appropriate reactants and reagents.

| Compound | | Spectroscopic |
|---|---|---|
| No. | Structure | Data |
| 23 | 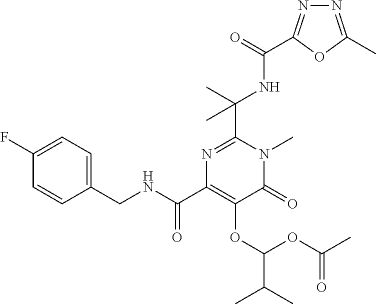 [SMILES: CC(=O)OC(Oc1c(C(=O)NCc2ccc(F)cc2)nc(C(C)(C)NC(=O)c2nnc(C)o2)n(C)c1=O)C(C)C] | (400 MHz, CDCl₃) δ 8.30 (s, 1H), 7.68 (d, J = 8.0 Hz, 1H), 7.36-7.37 (m, 2H), 7.00-7.05 (m, 2H), 6.51 (d, J = 6.4 Hz, 1H), 4.54-4.62 (m, 2H), 3.61 (s, 3H), 2.62 (s, 3H), 2.24-2.27 (m, 1H), 2.02-2.05 (m, 3H), 1.87 (s, 9H), 1.05-1.08 (m, 3H). LCMS anal. calcd. for C₂₆H₃₁FN₆O₇: 558.2; Found: 581.1 (M + 23)⁺ as (M + Na)⁺ |
| 24 | 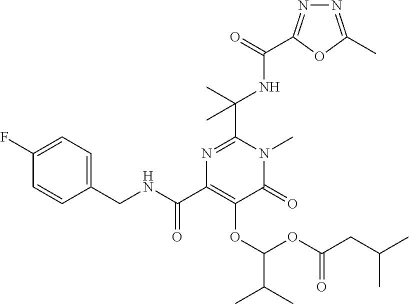 | (400 MHz, CDCl₃) δ 8.18 (s, 1H), 7.83 (s, 1H), 7.35~7.38 (m, 2H), 7.01~7.05 (m, 2H), 6.56 (d, J = 4.8 Hz, 1H), 4.59 (d, J = 6.0 Hz, 2H), 3.64 (s, 3H), 2.63 (s, 3H) 2.27~2.43 (m, 2H), 1.87~1.89 (m, 6H), 1.01~1.07 (m, 12H). LCMS anal. calcd. for C₂₈H₃₅FN₆O₇: 586.3; Found: 609.0 (M + 23)⁺ as (M + Na)⁺ |
| 25 | 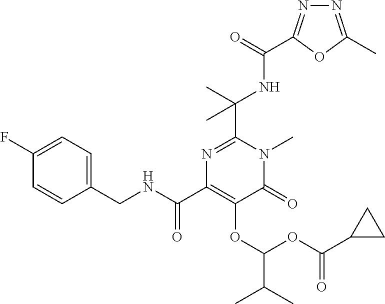 [SMILES: Cc1nnc(C(=O)NC(C)(C)c2nc(C(=O)NCc3ccc(F)cc3)c(OC(OC(=O)C3CC3)C(C)C)c(=O)n2C)o1] | (300 MHz, CDCl₃) δ 8.30 (s, 1H), 7.74-7.77 (m, 1H), 7.34-7.38 (m, 2H), 6.97- 7.02 (m, 2H), 6.54 (d, J = 6.0 Hz, 1H), 4.57-4.59 (m, 2H), 3.61 (s, 3H), 2.60 (s, 3H) 2.21-2.32 (m, 1H), 1.85-2.22 (m, 6H), 1.41- 1.45 (m, 1H), 095-1.06 (m, 6H), 0.88-0.90 (m, 4H). LCMS anal. calcd. for C₂₈H₃₃FN₆O₇: 584.2; Found: 607.2 (M + 23)⁺ as (M + Na)⁺ |
| 26 | 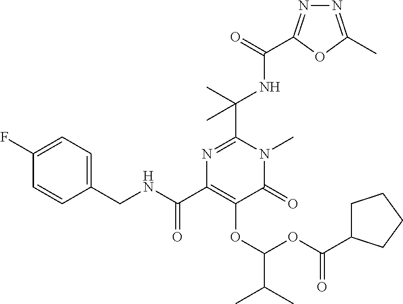 [SMILES: Cc1nnc(C(=O)NC(C)(C)c2nc(C(=O)NCc3ccc(F)cc3)c(OC(OC(=O)C3CCCC3)C(C)C)c(=O)n2C)o1] | (400 MHz, CDCl₃) δ 8.27 (s, 1H), 7.78 (s, 1H), 7.35- 7.36 (m, 2H), 6.99-7.04 (m, 2H), 6.58-6.60 (d, J = 6.0 Hz, 1H), 4.57-4.59 (m, 2H), 3.63 (s, 3H), 2.63 (s, 3H), 2.26-2.29 (m, 1H), 1.90- 1.92 (m, 6H), 1.60-1.69 (m, 9H), 1.04-1.08 (m, 6H). LCMS anal. calcd. for C₃₀H₃₇FN₆O₇: 612.3; Found: 635.3 (M + 23)⁺ as (M + Na)⁺ |
| 27 | 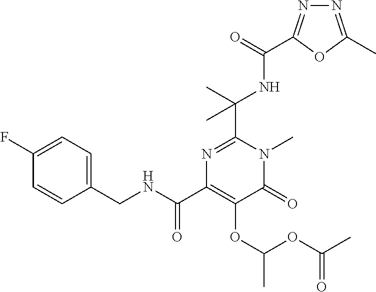 [SMILES: CC(=O)OC(C)Oc1c(C(=O)NCc2ccc(F)cc2)nc(C(C)(C)NC(=O)c2nnc(C)o2)n(C)c1=O] | (400 MHz, CDCl₃) δ 8.30 (s, 1H), 7.81 (s, 1H), 7.34- 7.37 (m, 2H), 6.99-7.04 (m, 2H), 6.57-6.58 (d, J = 5.2 Hz, 1H), 4.57 (s, 2H), 3.62 (s, 3H), 2.61 (s, 3H), 1.97 (s, 3H), 1.86-1.88 (m, 6H), 1.72 (d, J = 5.2 Hz, 3H). LCMS anal. calcd. for C₂₄H₂₇FN₆O₇: 530.2; Found: 553.2 (M + 23)⁺ as (M + Na)⁺ |
| 28 | 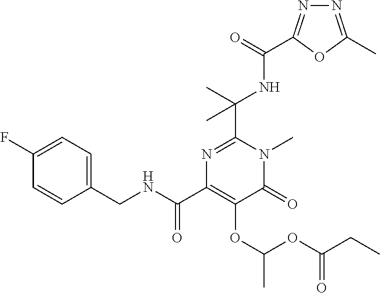 [SMILES: CCC(=O)OC(C)Oc1c(C(=O)NCc2ccc(F)cc2)nc(C(C)(C)NC(=O)c2nnc(C)o2)n(C)c1=O] | (400 MHz, CDCl₃) δ 8.31 (s, 1H), 7.83 (t, J = 5.6 Hz, 1H), 7.33-7.38 (m, 2H), 6.98-7.03 (m, 2H), 6.59- 6.63 (m, 1H), 4.57 (d, J = 4.4 Hz, 2H), 3.62 (s, 3H), 2.61 (s, 3H), 2.24-2.29 (m, 2H), 1.71-1.87 (m, 9H), 1.07 (m, 3H). LCMS anal. calcd. for C₂₅H₂₉FN₆O₇: 544.2; Found: 567.2 (M + 23)⁺ as (M + Na)⁺ |
| 29 | 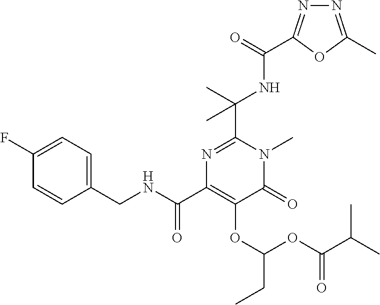 [SMILES: CCC(OC(=O)C(C)C)Oc1c(C(=O)NCc2ccc(F)cc2)nc(C(C)(C)NC(=O)c2nnc(C)o2)n(C)c1=O] | (400 MHz, CDCl₃) δ 8.25 (s, 1H), 7.79-7.83 (m, 1H), 7.35-7.38 (m, 2H), 6.99- 7.04 (m, 2H), 6.55-6.58 (m, 1H), 4.58 (d, J = 6.0 Hz, 2H), 3.63 (s, 3H), 2.48~2.63 (m, 4H) 1.99-2.15 (m, 2H), 1.86-1.89 (m, 6H), 1.04- 1.08 (m, 9H). LCMS anal. calcd. for C₂₇H₃₃FN₆O₇: 572.2; Found: 595.2 (M + 23)⁺ as (M + Na)⁺ |
| 30 | 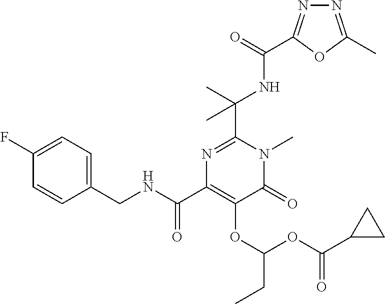 [SMILES: CCC(OC(=O)C1CC1)Oc1c(C(=O)NCc2ccc(F)cc2)nc(C(C)(C)NC(=O)c2nnc(C)o2)n(C)c1=O] | (300 MHz, CDCl₃) δ 8.39 (s, 1H), 7.79 (s, 1H), 7.25- 7.37 (m, 2H), 6.96-7.01 (m, 2H), 6.49-6.53 (m, 1H), 4.56 (d, J = 7.2 Hz, 2H), 3.59 (s, 3H), 2.58 (s, 3H), 1.81-1.85 (m, 6H), 1.12- 1.46 (m, 1H), 0.77-1.01 (m, 9H). LCMS anal. calcd. for C₂₇H₃₁FN₆O₇: 570.2; Found: 593.3 (M + 23)⁺ as (M + Na)⁺ |
| 31 | 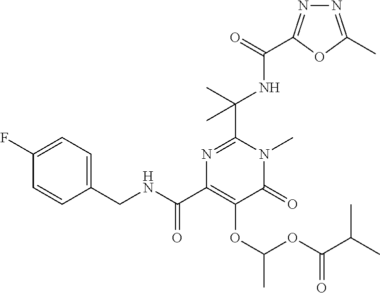 [SMILES: Cc1nnc(C(=O)NC(C)(C)c2nc(C(=O)NCc3ccc(F)cc3)c(OC(C)OC(=O)C(C)C)c(=O)n2C)o1] | (400 MHz, CDCl₃) δ 8.24 (s, 1H), 7.86 (s, 1H), 7.34- 7.36 (m, 2H), 6.99-7.03 (m, 2H), 6.63-6.64 (m, 1H), 4.57 (d, J = 6.0 Hz, 2H), 3.62 (s, 3H), 2.61 (s, 3H), 2.43-2.47 (m, 1H), 1.89 (d, J = 3.2 Hz, 6H), 1.73 (d, J = 5.2 Hz, 3H), 1.04-1.08 (m, 6H). LCMS anal. calcd. for C₂₆H₃₁FN₆O₇: 558.2; Found: 581.2 (M + 23)⁺ as (M + Na)⁺ |
| 32 | 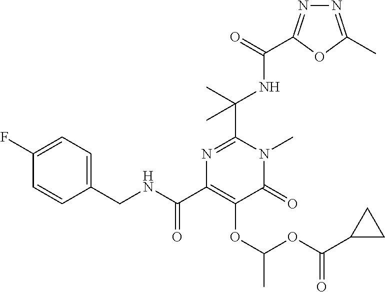 [SMILES: Cc1nnc(C(=O)NC(C)(C)c2nc(C(=O)NCc3ccc(F)cc3)c(OC(C)OC(=O)C3CC3)c(=O)n2C)o1] | (400 MHz, CDCl₃) δ 8.25 (s, 1H), 7.84 (t, J = 5.6 Hz, 1H), 7.35-7.36 (m, 2H), 6.98-7.03 (m, 2H), 6.62 (d, J = 5.2 Hz, 1H), 4.57 (d, J = 6.0 Hz, 2H), 3.62 (s, 3H), 2.63 (s, 3H), 1.88 (d, J = 2.4 Hz, 6H), 1.73 (d, J = 5.6 Hz, 3H), 1.47-1.50 (m, 1H), 0.79-0.94 (m, 4H). LCMS anal. calcd. for C₂₆H₂₉FN₆O₇: 556.2; Found: 557.2 (M + 1)⁺ |
| 33 | 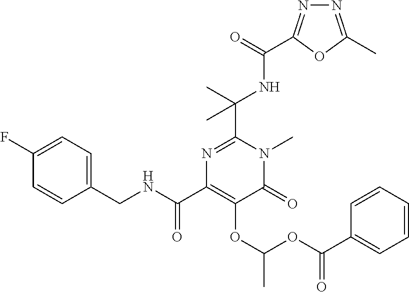 [SMILES: Cc1nnc(C(=O)NC(C)(C)c2nc(C(=O)NCc3ccc(F)cc3)c(OC(C)OC(=O)c3ccccc3)c(=O)n2C)o1] | (400 MHz, CDCl₃) δ 8.24 (s, 1H), 7.80-7.99 (m, 2H), 7.40-7.78 (m, 4H), 6.88- 7.29 (m, 5H), 4.48-4.51 (m, 2H), 3.63 (s, 3H), 2.63 (s, 3H), 1.87-1.89 (m, 3H), 1.57 (s, 6H). LCMS anal. calcd. for C₂₉H₂₉FN₆0₇: 592.2; Found: 615.2 (M + 23)⁺ as (M + Na)⁺ |
| 34 | 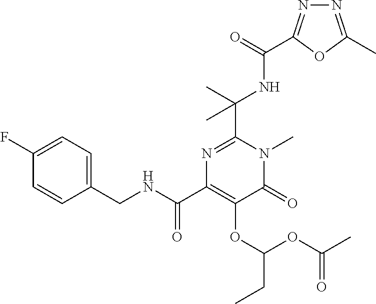 [SMILES: CCC(OC(C)=O)Oc1c(C(=O)NCc2ccc(F)cc2)nc(C(C)(C)NC(=O)c2nnc(C)o2)n(C)c1=O] | (400 MHz, CDCl₃) δ 8.21 (s, 1H), 7.75 (s, 1H), 7.32- 7.36 (m, 2H), 6.97-7.03 (m, 2H), 6.47-6.50 (d, J = 4.8 Hz, 1H), 4.54-4.58 (m, 2H), 3.61 (s, 3H), 2.63 (s, 3H), 2.11-2.13 (m, 2H), 1.97 (s, 3H), 1.88 (s, 6H), 0.99-1.02 (m, 3H). LCMS anal. calcd. for C₂₅H₂₉FN₆O₇: 544.2; Found: 567.2 (M + 23)⁺ as (M + Na)⁺ |
| 35 | 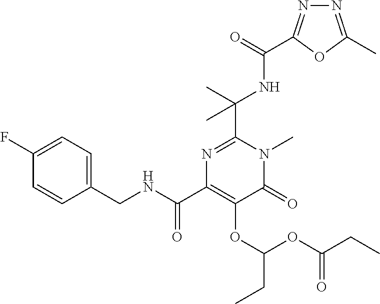 [SMILES: CCC(=O)OC(CC)Oc1c(C(=O)NCc2ccc(F)cc2)nc(C(C)(C)NC(=O)c2nnc(C)o2)n(C)c1=O] | (400 MHz, CDCl₃) δ 8.22 (s, 1H), 7.77 (s, 1H), 7.36- 7.37 (m, 2H), 7.02-7.04 (m, 2H), 6.55 (s, 1H), 4.58 (s, 2H), 3.63 (s, 3H), 2.64 (s, 3H), 1.60-2.16 (m, 10H), 1.01-1.04 (m, 6H). LCMS anal. calcd. for C₂₆H₃₁FN₆O₇: 558.2; Found: 581.2 (M + 23)⁺ as (M + Na)⁺ |
| 36 | 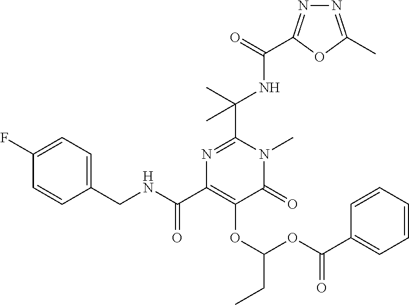 [SMILES: CCC(OC(=O)c1ccccc1)Oc1c(C(=O)NCc2ccc(F)cc2)nc(C(C)(C)NC(=O)c2nnc(C)o2)n(C)c1=O] | (300 MHz, CDCl₃) δ 8.22- 8.25 (m, 1H), 7.55-7.97 (m, 4H), 7.21-7.52 (m, 4H), 6.75-6.94 (m, 3H), 4.62- 4.73 (m, 2H), 3.62 (s, 3H), 2.62 (s, 3H), 2.21-2.28 (m, 2H), 1.88-1.87 (m, 6H), 1.09-1.15 (m, 3H). LCMS anal. calcd. for C₃₀H₃₁FN₆O₇: 606.2; Found: 629.3 (M + 23)⁺ as (M + Na)⁺ |
| 37 | 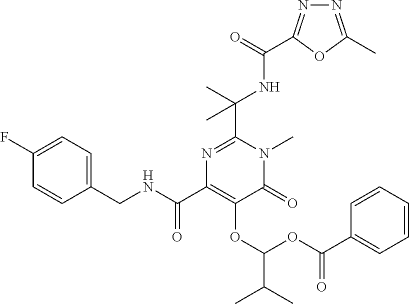 [SMILES: Cc1nnc(C(=O)NC(C)(C)c2nc(C(=O)NCc3ccc(F)cc3)c(OC(OC(=O)c3ccccc3)C(C)C)c(=O)n2C)o1] | (400 MHz, CDCl₃) δ 8.28 (s, 1H), 7.90-7.93 (d, J = 7.6 Hz, 2H), 7.16-7.71 (m, 6H), 6.82-6.94 (m, 3H), 4.45 (d, J = 5.6 Hz, 2H), 3.63 (s, 3H), 2.60 (s, 3H), 2.42-2.45 (m, 1H), 1.85- 1.87 (m, 6H), 1.13-1.19 (m, 6H). LCMS anal. calcd. for C₃₁H₃₃FN₆O₇: 620.2; Found: 643.1 (M + 23)⁺ as (M + Na)⁺ |
| 38 | 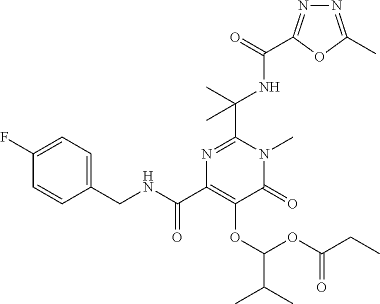 [SMILES: CCC(=O)OC(Oc1c(C(=O)NCc2ccc(F)cc2)nc(C(C)(C)NC(=O)c2nnc(C)o2)n(C)c1=O)C(C)C] | (400 MHz, CDCl₃) δ 8.30 (s, 1H), 7.72 (s, 1H), 7.34- 7.38 (m, 2H), 6.99-7.05 (m, 2H), 6.54-6.55 (d, J = 6.0 Hz, 1H), 4.56-4.60 (m, 2H), 3.63 (s, 3H), 2.62 (s, 3H), 2.21-2.23 (m, 3H), 1.87- 1.90 (m, 6H), 1.00-1.07 (m, 9H). LCMS anal. calcd. for C₂₇H₃₃FN₆O₇: 572.2; Found: 595.2 (M + 23)⁺ as (M + Na)⁺ |

### Example 3

### Metabolism of Compounds in Hepatocyte Suspensions

Selected compounds of the present invention (1 μM) were incubated with suspensions of cryopreserved hepatocytes from rat, dog and human subjects at a cell density of 1×10⁶ cells/mL. The samples were incubated for 0, 5, 15, 30, 60 and 90 minutes in a 95% humidified incubator at 37° C. with 5% CO₂. At each time point, the samples were quenched with acetonitrile containing an appropriate internal standard. The samples were then vortex-mixed and centrifuged at ˜2,900×g for 30 minutes. The supernatants were transferred to clean plates and analyzed using LC-MS/MS. Following quantification of prodrug and parent in the samples, half lives of conversion (t_{1/2}) were determined from the rates of prodrug degradation according to first-order kinetics. Conversion to active metabolite (compound 1a) was confirmed by following the formation of compound 1a.

TABLE 3
Prodrug hydrolysis in hepatocyte suspensions.

| | Remaining at 90 min | | | % Formation of 1a at 90 min | | |
|---|---|---|---|---|---|---|
| Compound | Rat | Dog | Human | Rat | Dog | Human |
| 1 | 30.30% | 15.94% | 19.32% | 60.95% | 68.13% | 82.32% |
| 7 | 2.72% | 3.31% | 2.12% | 88.70% | 73.50% | 17.30% |
| 12 | 38.75% | 5.97% | 14.81% | 16.24% | 10.64% | 31.65% |
| 15 | 6.20% | 8.90% | 14.00% | 44.48% | 55.44% | 59.40% |
| 20 | 0.60% | 1.70% | 1.80% | 82.60% | 98.61% | 75.97% |
| 22 | 3.58% | 3.21% | 4.27% | 90.16% | 97.16% | 116.59% |
| 23 | 50.65% | 8.14% | 28.06% | 28.12% | 54.98% | 48.93% |
| 24 | 13.13% | 4.81% | 16.34% | 34.02% | 25.07% | 28.10% |

Thus, the compounds of the present invention can act as prodrugs for active metabolites, such as compound 1a, which are known to be useful for the treatment of HIV infection.

### Example 4

### Rat Pharmacokinetic Studies

### Intravenous/Oral Dose:

Selected compounds of the present invention were administered in suitable vehicles to male Wistar-Hanover rats. Intravenous (IV) administration to rats was conducted via cannulas implanted in the jugular vein. For oral administration, test compounds were administered by gavage.

Blood samples were serially collected following dose administration for up to 24 hr and plasma was separated by centrifugation. The concentration of test compound in rat plasma were determined by a LC-MS/MS assay following a protein precipitation step and addition of an appropriate internal standard. Quantification was done by determining peak area-ratios of the test compound and compound 1a (the metabolite of each of the test compounds) to the internal standard.

Pharmacokinetic parameters were obtained using non-compartmental methods (Watson®). The area under the plasma concentration-time curve (AUC₀₋ₜ) was calculated from the first time point (0 min) up to the last time point with measurable test compound concentration using the linear trapezoidal or linear/log-linear trapezoidal rule. The IV plasma clearance was calculated by dividing the dose by AUC_{0-inf}. The terminal half-life of elimination was determined by unweighted linear regression analysis of the log-transformed data. The time points for determination of half-life were selected by visual inspection of the data. The volume of distribution at steady state (Vdₛₛ) was obtained from the product of plasma clearance and mean residence time (determined by dividing the area under the first moment curve by the area under the curve). The maximum plasma concentration (Cₘₐₓ) and the time at which maximum concentration occurred (Tₘₐₓ) were obtained by inspection of the plasma concentration-time data. Absolute oral bioavailability was determined from dose-adjusted IV and P.O. AUC ratios.

TABLE 4
Intravenous pharmacokinetics in Wistar-Hanover rats

| | AUC₀₋ₓ | nAUC_{0-∞} | Clₚ | Vdₛₛ | t_{1/2} |
|---|---|---|---|---|---|
| Compound | (μM · hr) | (μM · hr) | (mL/min/kg) | (L/kg) | (hr) |
| 1 | 0.012 | 0.012 | ND | ND | 0.15 |
| | (0.38) | (0.47) | | | |
| 2 | 0.119 | 0.119 | 244 | 2.17 | 0.17 |
| | (0.622) | (0.784) | | | |
| 15 | 0.085 | 0.085 | 388 | 3.48 | 0.14 |
| | (0.647) | (0.838) | | | |
| 22 | 0.012 | 0.012 | ND | ND | 0.04 |
| | (0.304) | (0.24) | | | |

Numbers in parenthesis represent parameters for 1a following administration of the prodrug
ND = Not determined due to insufficient data;
BLQ = Below the limit of quantification

TABLE 5
Oral pharmacokinetics in Wistar-Hanover rats

| | AUC0-x hr | nAUC | Cmax | Tmax | |
|---|---|---|---|---|---|
| Compound | (μM · hr) | (μM · hr) | (μM) | (hr) | F (%)* |
| 1 | 0.002 | 0.0004 | 0.006 | 0.25 | 3 |
| | (0.603) | (0.152) | (0.31) | (1) | (15.7) |
| 2 | 0.039 | 0.008 | 0.019 | 1.13 | 6.6 |
| | (2.48) | (0.642) | (1.27) | (0.5) | (66) |
| 15 | 0.028 | 0.006 | 0.021 | 0.75 | 7 |
| | (1.52) | (0.394) | (0.685) | (0.38) | (40) |
| 22 | ND | ND | BLQ | ND | ND |
| | (1.17) | (0.294) | (1.38) | | (30) |

Numbers in parenthesis represent parameters for 1a following administration of the prodrug
ND = Not determined due to insufficient data;
BLQ = Below the limit of quantification
*Bioavailability for 1a (numbers in parenthesis) were obtained from the ratios of the normalized oral AUC after prodrug administration to the normalized IV AUC obtained from dosing 1a to rats.

### Example 5

### Dog Pharmacokinetic Studies

### Oral/Colonic Dose:

Six Male Beagle Dogs (Marshall Farms) weighing 9.0-11.6 kg were used for the studies. Studies were conducted under a protocol approved by the WP-IACUC (Animal Procedure Statement #09068544080285). Following overnight-fasting, dogs were dosed either orally (N=3) or colonically via a retrograde catheter method (N=3) with 4 mg/mL solution at 1 mpk of test compound. Dosing was followed by 5 mL water rinse via oral gavage for oral dosing or with a post-dose water flush of the catheter for colonic dosing. Water was restricted for 1 hour post dose. Food was returned at 4 hours after dosing. Blood (1-mL) was drawn at pre-dose, 0.25, 0.5, 1, 2, 4, 6, 8 and 24 hours post-dosing. The plasma was separated by centrifugation (10 minutes at 2500 g) and kept frozen at −70° C. until analysis by LC-MS/MS.

TABLE 6
Mean [±SE] pharmacokinetic parameters for 1 and 22 and 1a after administration
of 1, 22 or 1a in appropriate vehicles to fasted Beagle dogs at 4 mpk.

| Compound | Dosing | Compound | AUC_{0-24 hr} | Cₘₐₓ | | AUC |
|---|---|---|---|---|---|---|
| Dosed | Route | Measured | (μM · hr) | (μM) | Tₘₐₓ* (hr) | ratio |
| 1 | Oral | 1 | 0.146 ± 0.033 | 0.212 ± 0.032 | 0.3 | — |
| | | | | | (0.3-0.5) | |
| 1 | Colonic | 1 | 0.083 ± 0.034 | 0.091 ± 0.025 | 0.3 | — |
| 1 | Oral | 1a | 7.646 ± 0.481 | 2.763 ± 0.364 | 1.0 | — |
| | | | | | (0.5-1.0) | |
| 1 | Colonic | 1a | 3.053 ± 0.840 | 0.966 ± 0.171 | 0.5 | 0.39 |
| | | | | | (0.3-1.0) | |
| 22 | Oral | 22 | 0.006 ± 0.002 | 0.008 ± 0.002 | 0.5 | — |
| | | | | | (0.25-0.5) | |
| 22 | Colonic | 22 | 0.014 ± 0.003 | 0.010 ± 0.003 | 0.25 | — |
| 22 | Oral | 1a | 9.593 ± 0.577 | 3.325 ± 0.830 | 1.0 | — |
| | | | | | (0.5-2.0) | |
| 22 | Colonic | 1a | 2.902 ± 0.287 | 0.684 ± 0.0.46 | 1.0 | 0.30 |
| 1a (metabolite | Oral | 1a | 11.687 ± 1.949 | 5.580 ± 0.990 | 0.5 | — |
| of cpds 1 and 22) | | | | | (0.3-0.5) | |
| 1a (metabolite | Colonic | 1a | 1.204 ± 1.055 | 0.224 ± 0.172 | 0.5 | 0.10 |
| of cpds 1 and 22) | | | | | (0.3-2.0) | |

*For Tₘₐₓ, median value is provided

This data indicates that Compounds 1 and 22, which are representative compounds of the present invention, show an unexpected and significant increase in colonic absorption versus their metabolite, Compound 1a.

### Uses of the Amido-Substituted Pyrimidinone Derivatives

The Amido-Substituted Pyrimidinone Derivatives are useful in human and veterinary medicine for treating or preventing HIV infection in a subject. In one embodiment, the Amido-Substituted Pyrimidinone Derivatives can be inhibitors of HIV viral replication. In a specific embodiment, the Amido-Substituted Pyrimidinone Derivatives are inhibitors of HIV-1. Accordingly, the Amido-Substituted Pyrimidinone Derivatives are useful for treating HIV infections and AIDS. In accordance with the invention, the Amido-Substituted Pyrimidinone Derivatives can be administered to a subject in need of treatment or prevention of HIV infection.

Accordingly, in one embodiment, the invention provides methods for treating HIV infection in a subject comprising administering to the subject an effective amount of at least one Amido-Substituted Pyrimidinone Derivative or a pharmaceutically acceptable salt thereof. In a specific embodiment, the present invention provides methods for treating AIDS in a subject comprising administering to the subject an effective amount of at least one Amido-Substituted Pyrimidinone Derivative or a pharmaceutically acceptable salt thereof

### Treatment or Prevention of HIV Infection

The Amido-Substituted Pyrimidinone Derivatives are useful in the inhibition of HIV, the treatment of HIV infection and/or reduction of the likelihood or severity of symptoms of HIV infection and the inhibition of HIV viral replication and/or HIV viral production in a cell-based system. For example, the Amido-Substituted Pyrimidinone Derivatives are useful in treating infection by HIV after suspected past exposure to HIV by such means as blood transfusion, exchange of body fluids, bites, accidental needle stick, or exposure to subject blood during surgery or other medical procedures.

In one embodiment, the HIV infection has progressed to AIDS.

Accordingly, in one embodiment, the invention provides methods for treating HIV infection in a subject, the methods comprising administering to the subject an effective amount of at least one Amido-Substituted Pyrimidinone Derivative or a pharmaceutically acceptable salt thereof. In a specific embodiment, the amount administered is effective to treat or prevent infection by HIV in the subject. In another specific embodiment, the amount administered is effective to inhibit HIV viral replication and/or viral production in the subject.

The Amido-Substituted Pyrimidinone Derivatives are also useful in the preparation and execution of screening assays for antiviral compounds. For example the Amido-Substituted Pyrimidinone Derivatives are useful for identifying resistant HIV cell lines harboring mutations, which are excellent screening tools for more powerful antiviral compounds. Furthermore, the Amido-Substituted Pyrimidinone Derivatives are useful in establishing or determining the binding site of other antivirals to the HIV Integrase.

The compositions and combinations of the present invention can be useful for treating a subject suffering from infection related to any HIV genotype.

### Combination Therapy

In another embodiment, the present methods for treating or preventing HIV infection can further comprise the administration of one or more additional therapeutic agents which are not Amido-Substituted Pyrimidinone Derivatives.

In one embodiment, the additional therapeutic agent is an antiviral agent.

In another embodiment, the additional therapeutic agent is an immunomodulatory agent, such as an immunosuppressive agent.

Accordingly, in one embodiment, the present invention provides methods for treating a viral infection in a subject, the method comprising administering to the subject: (i) at least one Amido-Substituted Pyrimidinone Derivative (which may include two or more different Amido-Substituted Pyrimidinone Derivatives), or a pharmaceutically acceptable salt thereof, and (ii) at least one additional therapeutic agent that is other than a Amido-Substituted Pyrimidinone Derivative, wherein the amounts administered are together effective to treat or prevent a viral infection.

When administering a combination therapy of the invention to a subject, therapeutic agents in the combination, or a pharmaceutical composition or compositions comprising therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for non-limiting illustration purposes, a Amido-Substituted Pyrimidinone Derivative and an additional therapeutic agent may be present in fixed amounts (dosage amounts) in a single dosage unit (e.g., a capsule, a tablet and the like).

In one embodiment, the at least one Amido-Substituted Pyrimidinone Derivative is administered during a time when the additional therapeutic agent(s) exert their prophylactic or therapeutic effect, or vice versa.

In another embodiment, the at least one Amido-Substituted Pyrimidinone Derivative and the additional therapeutic agent(s) are administered in doses commonly employed when such agents are used as monotherapy for treating a viral infection.

In another embodiment, the at least one Amido-Substituted Pyrimidinone Derivative and the additional therapeutic agent(s) are administered in doses lower than the doses commonly employed when such agents are used as monotherapy for treating a viral infection.

In still another embodiment, the at least one Amido-Substituted Pyrimidinone Derivative and the additional therapeutic agent(s) act synergistically and are administered in doses lower than the doses commonly employed when such agents are used as monotherapy for treating a viral infection.

In one embodiment, the at least one Amido-Substituted Pyrimidinone Derivative and the additional therapeutic agent(s) are present in the same composition. In one embodiment, this composition is suitable for oral administration. In another embodiment, this composition is suitable for intravenous administration. In another embodiment, this composition is suitable for subcutaneous administration. In still another embodiment, this composition is suitable for parenteral administration.

Viral infections and virus-related disorders that can be treated or prevented using the combination therapy methods of the present invention include, but are not limited to, those listed above.

In one embodiment, the viral infection is HIV infection.

In another embodiment, the viral infection is AIDS.

The at least one Amido-Substituted Pyrimidinone Derivative and the additional therapeutic agent(s) can act additively or synergistically. A synergistic combination may allow the use of lower dosages of one or more agents and/or less frequent administration of one or more agents of a combination therapy. A lower dosage or less frequent administration of one or more agents may lower toxicity of therapy without reducing the efficacy of therapy.

In one embodiment, the administration of at least one Amido-Substituted Pyrimidinone Derivative and the additional therapeutic agent(s) may inhibit the resistance of a viral infection to these agents.

As noted above, the present invention is also directed to use of a compound of Formula I with one or more anti-HIV agents. An “anti-HIV agent” is any agent which is directly or indirectly effective in the inhibition of HIV reverse transcriptase or another enzyme required for HIV replication or infection, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, immunomodulators, antiinfectives, or vaccines useful for treating HIV infection or AIDS. Suitable HIV antivirals for use in combination with the compounds of the present invention include, for example, those listed in Table A as follows:

TABLE A

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| Dolutegravir | PI |
| efavirenz, EFV, Sustiva ®, Stocrin ® | nnRTI |
| efavirenz + emtricitabine + tenofovir DF, Atripla ® | nnRTI + |
| | nRTI |
| Elvitegravir | InI |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125 | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| raltegravir, MK-0518, Isentress ® | InI |
| rilpivirine, TMC-278 | nnRTI |
| ritonavir, Norvir ® | PI |
| saquinavir, Invirase ®, Fortovase ® | PI |
| stavudine, d4T, didehydrodeoxythymidine, Zerit ® | nRTI |
| tenofovir DF (DF = disoproxil fumarate), TDF, | nRTI |
| Viread ® | |
| tipranavir, Aptivus ® | PI |

EI = entry inhibitor; FI = fusion inhibitor; InI = integrase inhibitor; PI = protease inhibitor; nRTI = nucleoside reverse transcriptase inhibitor; nnRTI = non-nucleoside reverse transcriptase inhibitor.
Some of the drugs listed in the table are used in a salt form; e.g., abacavir sulfate, indinavir sulfate, atazanavir sulfate, nelfinavir mesylate.

In one embodiment, the one or more anti-HIV drugs are selected from raltegravir, lamivudine, abacavir, ritonavir, dolutegravir, darunavir, atazanavir, emtricitabine, tenofovir, elvitegravir, rilpivirine and lopinavir.

In another embodiment, the compound of formula (I) is used in combination with a single anti-HIV drug which is raltegravir.

In another embodiment, the compound of formula (I) is used in combination with a single anti-HIV drug which is lamivudine.

In still another embodiment, the compound of formula (I) is used in combination with a single anti-HIV drug which is atazanavir.

In another embodiment, the compound of formula (I) is used in combination with a single anti-HIV drug which is darunavir.

In another embodiment, the compound of formula (I) is used in combination with a single anti-HIV drug which is rilpivirine.

In yet another embodiment, the compound of formula (I) is used in combination with a single anti-HIV drug which is dolutegravir.

In another embodiment, the compound of formula (I) is used in combination with a single anti-HIV drug which is elvitegravir.

In one embodiment, the compound of formula (I) is used in combination with two anti-HIV drugs which are lamivudine and abacavir.

In another embodiment, the compound of formula (I) is used in combination with two anti-HIV drugs which are darunavir and raltegravir.

In another embodiment, the compound of formula (I) is used in combination with two anti-HIV drugs which are emtricitabine and tenofovir.

In still another embodiment, the compound of formula (I) is used in combination with two anti-HIV drugs which are atazanavir and raltegravir.

In another embodiment, the compound of formula (I) is used in combination with two anti-HIV drugs which are ritonavir and lopinavir.

In another embodiment, the compound of formula (I) is used in combination with two anti-HIV drugs which are lamivudine and raltegravir.

In one embodiment, the compound of formula (I) is used in combination with three anti-HIV drug which are abacavir, lamivudine and raltegravir.

In another embodiment, the compound of formula (I) is used in combination with three anti-HIV drug which are lopinavir, ritonavir and raltegravir.

In one embodiment, the present invention provides pharmaceutical compositions comprising (i) a compound of formula (I) or a pharmaceutically acceptable salt thereof; (ii) a pharmaceutically acceptable carrier; and (iii) one or more additional anti-HIV agents selected from lamivudine, abacavir, ritonavir and lopinavir, or a pharmaceutically acceptable salt thereof, wherein the amounts present of components (i) and (iii) are together effective for the treatment or prophylaxis of infection by HIV or for the treatment, prophylaxis, or delay in the onset or progression of AIDS in the subject in need thereof.

In another embodiment, the present invention provides a method for the treatment or prophylaxis of infection by HIV or for the treatment, prophylaxis, or delay in the onset or progression of AIDS in a subject in need thereof, which comprises administering to the subject (i) a compound of formula (I) or a pharmaceutically acceptable salt thereof and (ii) one or more additional anti-HIV agents selected from lamivudine, abacavir, ritonavir and lopinavir, or a pharmaceutically acceptable salt thereof, wherein the amounts administered of components (i) and (ii) are together effective for the treatment or prophylaxis of infection by HIV or for the treatment, prophylaxis, or delay in the onset or progression of AIDS in the subject in need thereof

It is understood that the scope of combinations of the compounds of this invention with anti-HIV agents is not limited to the HIV antivirals listed in Table A, but includes in principle any combination with any pharmaceutical composition useful for the treatment or prophylaxis of AIDS. The HIV antiviral agents and other agents will typically be employed in these combinations in their conventional dosage ranges and regimens as reported in the art, including, for example, the dosages described in the Physicians' Desk Reference, Thomson PDR, Thomson PDR, 57^{th} edition (2003), the 58^{th} edition (2004), the 59^{th} edition (2005), and the like. The dosage ranges for a compound of the invention in these combinations are the same as those set forth above.

The compounds of this invention are also useful in the preparation and execution of screening assays for antiviral compounds. For example, the compounds of this invention are useful for isolating enzyme mutants, which are excellent screening tools for more powerful antiviral compounds. Furthermore, the compounds of this invention are useful in establishing or determining the binding site of other antivirals to HIV integrase, e.g., by competitive inhibition. Thus the compounds of this invention are commercial products to be sold for these purposes.

The doses and dosage regimen of the other agents used in the combination therapies of the present invention for the treatment or prevention of HIV infection can be determined by the attending clinician, taking into consideration the approved doses and dosage regimen in the package insert; the age, sex and general health of the subject; and the type and severity of the viral infection or related disease or disorder. When administered in combination, the Amido-Substituted Pyrimidinone Derivative(s) and the other agent(s) can be administered simultaneously (i.e., in the same composition or in separate compositions one right after the other) or sequentially. This particularly useful when the components of the combination are given on different dosing schedules, e.g., one component is administered once daily and another component is administered every six hours, or when the pharmaceutical compositions are different, e.g., one is a tablet and one is a capsule. A kit comprising the separate dosage forms is therefore advantageous.

### Compositions and Administration

When administered to a subject, the Amido-Substituted Pyrimidinone Derivatives can be administered as a component of a composition that comprises a pharmaceutically acceptable carrier or vehicle. The present invention provides pharmaceutical compositions comprising an effective amount of at least one Amido-Substituted Pyrimidinone Derivative and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

Additionally, the compositions of the present invention may be formulated in sustained release form to provide the rate controlled release of any one or more of the components or active ingredients to optimize therapeutic effects, i.e., antiviral activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the one or more Amido-Substituted Pyrimidinone Derivatives are administered orally.

In another embodiment, the one or more Amido-Substituted Pyrimidinone Derivatives are administered intravenously.

In one embodiment, a pharmaceutical preparation comprising at least one Amido-Substituted Pyrimidinone Derivative is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1% to about 99% of the Amido-Substituted Pyrimidinone Derivative(s) by weight or volume. In various embodiments, the present compositions can contain, in one embodiment, from about 1% to about 70% or from about 5% to about 60% of the Amido-Substituted Pyrimidinone Derivative(s) by weight or volume.

The compounds of Formula I can be administered orally in a dosage range of 0.001 to 1000 mg/kg of mammal (e.g., human) body weight per day in a single dose or in divided doses. One dosage range is 0.01 to 500 mg/kg body weight per day orally in a single dose or in divided doses. Another dosage range is 0.1 to 100 mg/kg body weight per day orally in single or divided doses. For oral administration, the compositions can be provided in the form of tablets or capsules containing 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the subject to be treated. The specific dose level and frequency of dosage for any particular subject may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

The amount and frequency of administration of the Amido-Substituted Pyrimidinone Derivatives will be regulated according to the judgment of the attending clinician considering such factors as age, condition and size of the subject as well as severity of the symptoms being treated. The compositions of the invention can further comprise one or more additional therapeutic agents, selected from those listed above herein. Accordingly, in one embodiment, the present invention provides compositions comprising: (i) at least one Amido-Substituted Pyrimidinone Derivative or a pharmaceutically acceptable salt thereof; (ii) one or more additional therapeutic agents that are not a Amido-Substituted Pyrimidinone Derivative; and (iii) a pharmaceutically acceptable carrier, wherein the amounts in the composition are together effective to treat HIV infection.

### Kits

In one aspect, the present invention provides a kit comprising a therapeutically effective amount of at least one Amido-Substituted Pyrimidinone Derivative, or a pharmaceutically acceptable salt of said compound and a pharmaceutically acceptable carrier, vehicle or diluent.

In another aspect the present invention provides a kit comprising an amount of at least one Amido-Substituted Pyrimidinone Derivative, or a pharmaceutically acceptable salt of said compound and an amount of at least one additional therapeutic agent listed above, wherein the amounts of the two or more active ingredients result in a desired therapeutic effect. In one embodiment, the one or more Amido-Substituted Pyrimidinone Derivatives and the one or more additional therapeutic agents are provided in the same container. In one embodiment, the one or more Amido-Substituted Pyrimidinone Derivatives and the one or more additional therapeutic agents are provided in separate containers.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

1. A compound having the formula (I): 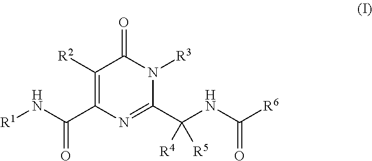
or a pharmaceutically acceptable salt thereof,
wherein:
R¹ is selected from —(C₁-C₆ alkylene)-(C₆-C₁₀ aryl), —(C₁-C₆ alkylene)-(5 or 6-membered monocyclic heteroaryl) or —(C₁-C₆ alkylene)-(9 or 10-membered bicyclic heteroaryl, wherein said C₆-C₁₀ aryl group, said 5 or 6-membered monocyclic heteroaryl group and said 9 or 10-membered bicyclic heteroaryl group can be optionally substituted with up to four R⁷ groups, which can be the same or different;
R² is —O—(C₁-C₆ alkylene)-X—C(O)—Y—R⁹;
R³ is H or C₁-C₆ alkyl;
R⁴ is H or C₁-C₆ alkyl;
R⁵ is H or C₁-C₆ alkyl;
R⁶ is selected from C₆-C₁₀ aryl, 5 or 6-membered monocyclic heteroaryl and 9 or 10-membered bicyclic heteroaryl, any of which can be optionally substituted with up to four R⁷ groups;
R⁷ is C₁-C₆ alkyl, 5 or 6-membered heterocycloalkyl, C₁-C₆ hydroxyalkyl, C₁-C₆ haloalkyl, halo, —CN, —N(R⁸)₂, —CH₂N(R⁸)₂, —OR⁸, —C(O)OR⁸, —SR⁸, —S(O)₂R⁸ or —C(O)N(R⁸)₂, wherein said 5 or 6-membered heterocycloalkyl group can be optionally substituted with a group selected from C₁-C₆ alkyl, halo, C₁-C₆ hydroxyalkyl, C₁-C₆ haloalkyl, halo, —CN, —N(R⁸)₂ and —OR⁸;
each occurrence of R⁸ is independently H or C₁-C₆ alkyl;
R⁹ is selected from C₁-C₆ alkyl, C₆-C₁₀ aryl, 5 or 6-membered monocyclic heteroaryl, 9 or 10-membered bicyclic heteroaryl, 3 to 7-membered monocyclic heterocycloalkyl, 8 to 10-membered bicyclic heterocycloalkyl and C₃-C₆ cycloalkyl, wherein said C₁-C₆ alkyl group can be optionally substituted with R¹⁰, and wherein said C₆-C₁₀ aryl group, 5 or 6-membered monocyclic heteroaryl group, 9 or 10-membered bicyclic heteroaryl group, 3 to 7-membered monocyclic heterocycloalkyl group, 8 to 10-membered bicyclic heterocycloalkyl group and C₃-C₆ cycloalkyl group can be optionally substituted with up to four R⁷ groups, which can be the same or different;
R¹⁰ is selected from C₆-C₁₀ aryl, 5 or 6-membered monocyclic heteroaryl, 9 or 10-membered bicyclic heteroaryl, 3 to 7-membered monocyclic heterocycloalkyl, 8 to 10-membered bicyclic heterocycloalkyl and C₃-C₆ cycloalkyl, wherein said C₆-C₁₀ aryl group, 5 or 6-membered monocyclic heteroaryl group, 9 or 10-membered bicyclic heteroaryl group, 3 to 7-membered monocyclic heterocycloalkyl group, 8 to 10-membered bicyclic heterocycloalkyl group and C₃-C₆ cycloalkyl group can be optionally substituted with up to four R⁷ groups, which can be the same or different;
X is O or NH; and
Y is a bond, O or NH.

2. The compound of claim 1, wherein R¹ is benzyl, and wherein the phenyl moiety of said benzyl group is optionally substituted with one substituent.

3. The compound of claim 1, wherein R⁶ is 5-membered monocyclic heteroaryl, which can be optionally substituted with one substituent.

4. The compound of claim 1, wherein R³ is C₁-C₆ alkyl.

5. The compound of claim 1, wherein at least one of R⁴ and R⁵ is C₁-C₆ alkyl.

6. The compound of claim 1, wherein R³, R⁴ and R⁵ are each methyl.

7. The compound of claim 1, having the formula (Ia): 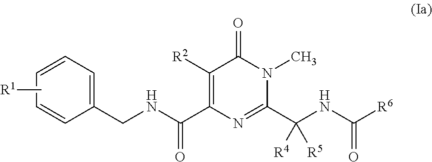
or a pharmaceutically acceptable salt thereof,
wherein:
Y is a bond or O;
R¹ is halo;
R² is —O—(C₁-C₄ alkylene)-O—C(O)—Y—R⁹;
R⁴ is C₁-C₃ alkyl;
R⁵ is C₁-C₃ alkyl;
R⁶ is 5-membered monocyclic heteroaryl, which can be optionally substituted with C₁-C₆ alkyl; and
R⁹ is selected from C₁-C₆ alkyl, C₆-C₁₀ aryl, and C₃-C₆ cycloalkyl, wherein said C₁-C₆ alkyl group can be optionally substituted with phenyl or C₃-C₆ cycloalkyl.

8. The compound of claim 1, having the formula (Ib): 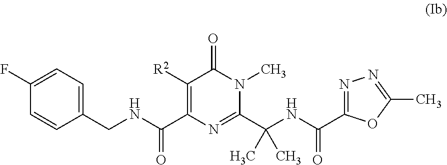
or a pharmaceutically acceptable salt thereof,
wherein:
R² is —O—(CHR⁷)—O—C(O)—Y—R⁹;
Y is O or a bond;
R⁷ is H, methyl, ethyl or isopropyl;
R⁹ is C₁-C₄ alkyl, —(CH₂)ₙ—C₃-C₆ cycloalkyl or —(CH₂)ₙ-phenyl; and
n is 0 or 1.

9. The compound of claim 1, wherein R² is —O—(C₁-C₄ alkylene)-O—C(O)—O—R⁹.

10. The compound of claim 8, wherein R² is —O—(C₁-C₄ alkylene)-O—C(O)—R⁹.

11. The compound of claim 8 wherein R² is selected from:

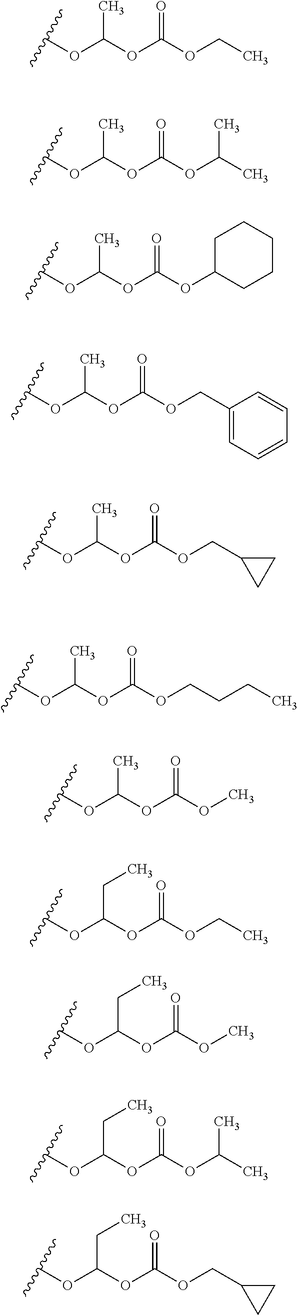

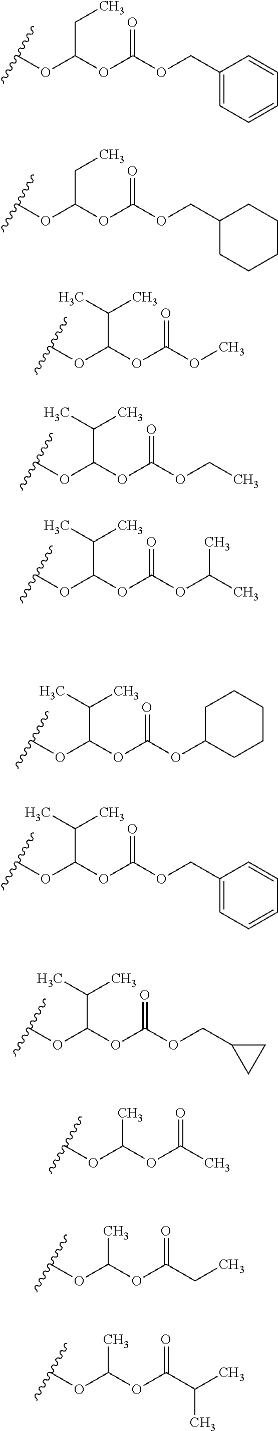

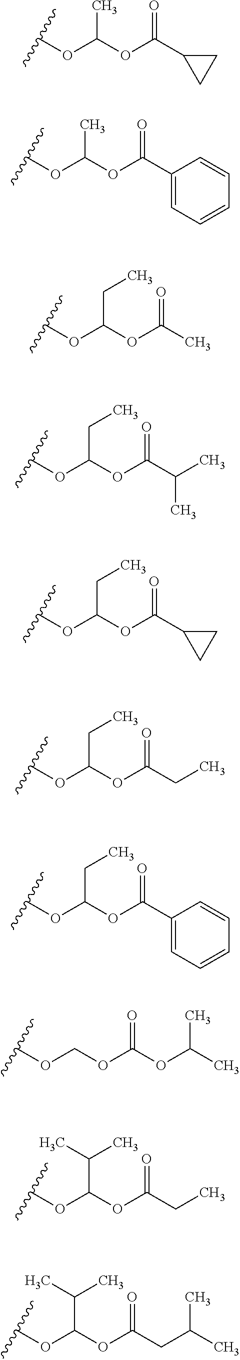

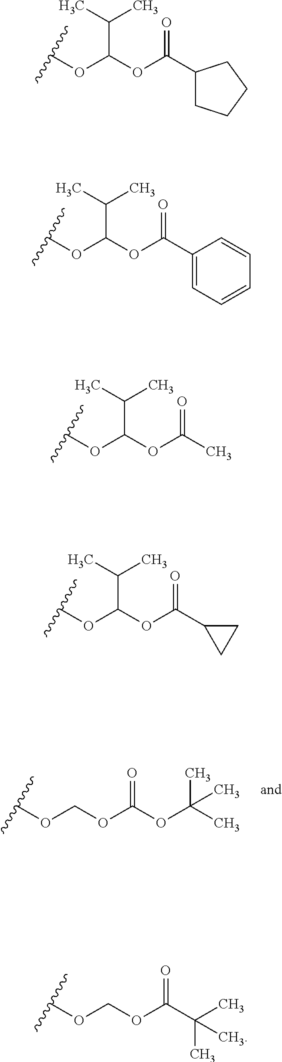

12. The compound of claim 11, wherein R² is selected from:

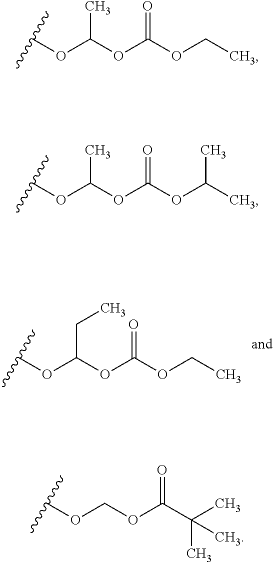

13. The compound of claim 1 having the structure 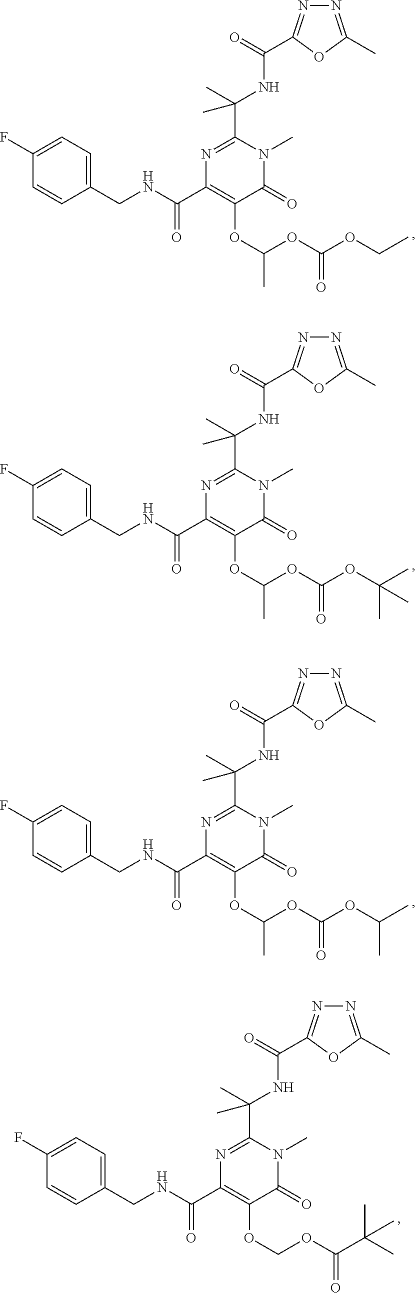 [SMILES: CCOC(=O)OC(C)Oc1c(C(=O)NCc2ccc(F)cc2)nc(C(C)(C)NC(=O)c2nnc(C)o2)n(C)c1=O ; Cc1nnc(C(=O)NC(C)(C)c2nc(C(=O)NCc3ccc(F)cc3)c(OC(C)OC(=O)OC(C)(C)C)c(=O)n2C)o1 ; Cc1nnc(C(=O)NC(C)(C)c2nc(C(=O)NCc3ccc(F)cc3)c(OC(C)OC(=O)OC(C)C)c(=O)n2C)o1 ; Cc1nnc(C(=O)NC(C)(C)c2nc(C(=O)NCc3ccc(F)cc3)c(OCOC(=O)C(C)(C)C)c(=O)n2C)o1] 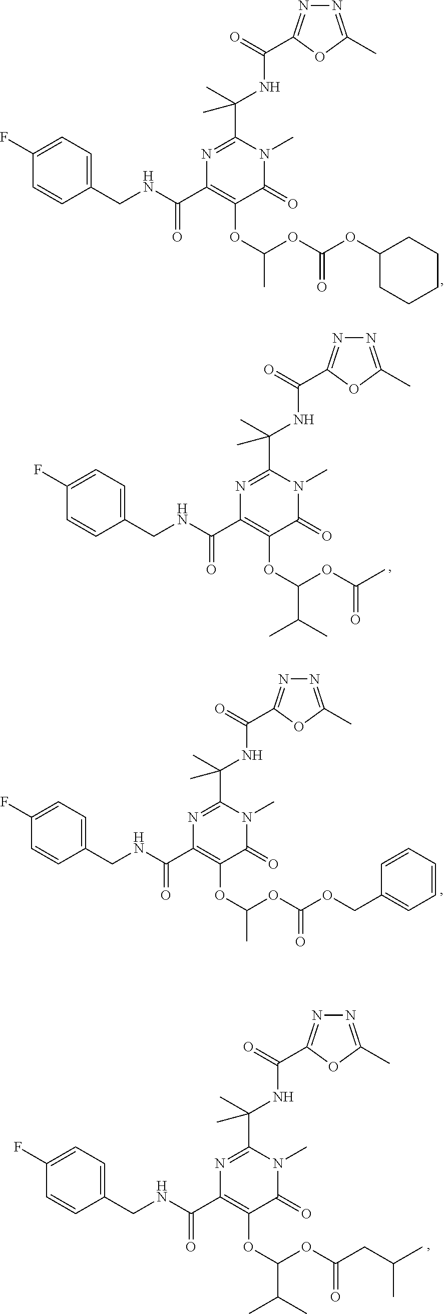 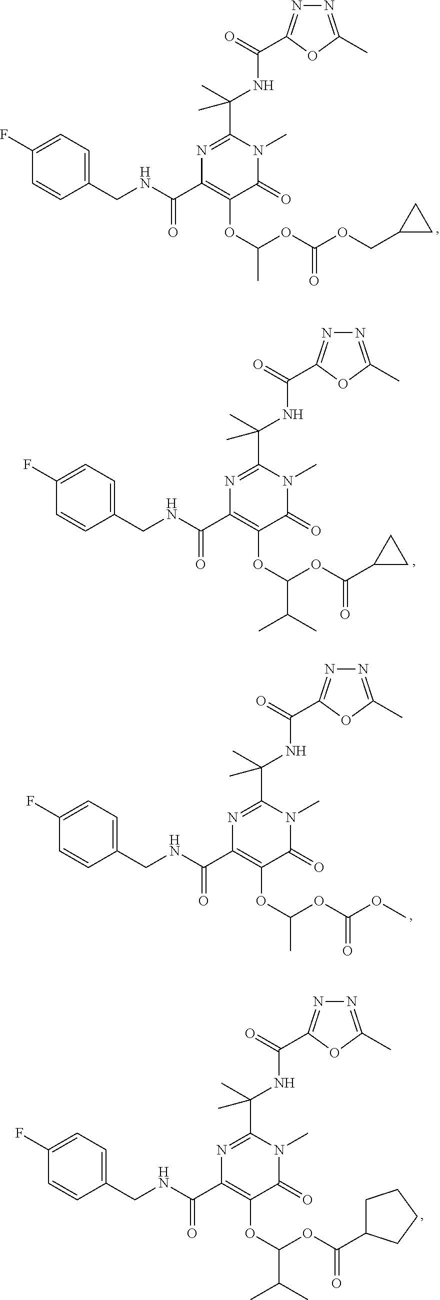 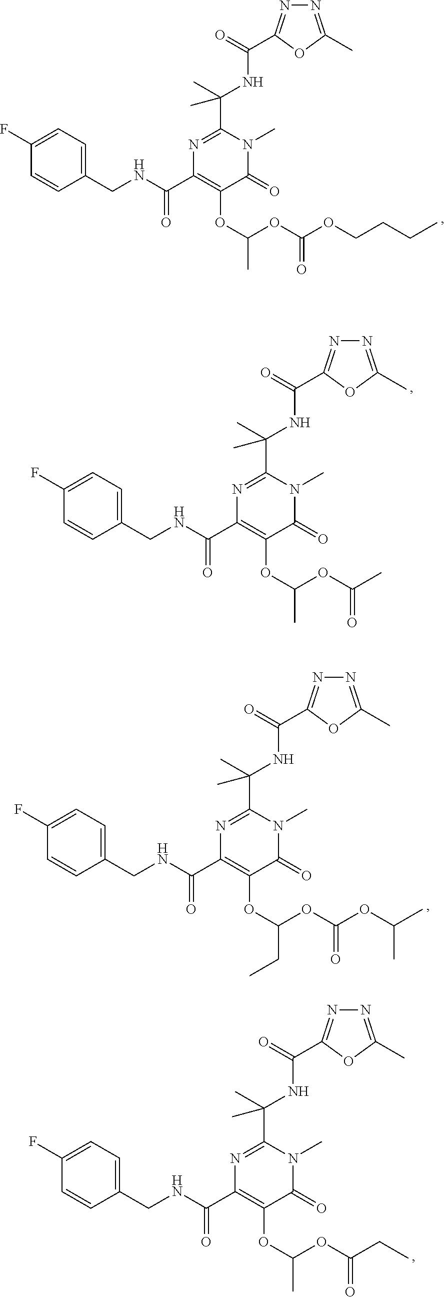 [SMILES: CCCCOC(=O)OC(C)Oc1c(C(=O)NCc2ccc(F)cc2)nc(C(C)(C)NC(=O)c2nnc(C)o2)n(C)c1=O ; CC(=O)OC(C)Oc1c(C(=O)NCc2ccc(F)cc2)nc(C(C)(C)NC(=O)c2nnc(C)o2)n(C)c1=O ; CCC(OC(=O)OC(C)C)Oc1c(C(=O)NCc2ccc(F)cc2)nc(C(C)(C)NC(=O)c2nnc(C)o2)n(C)c1=O ; CCC(=O)OC(C)Oc1c(C(=O)NCc2ccc(F)cc2)nc(C(C)(C)NC(=O)c2nnc(C)o2)n(C)c1=O] 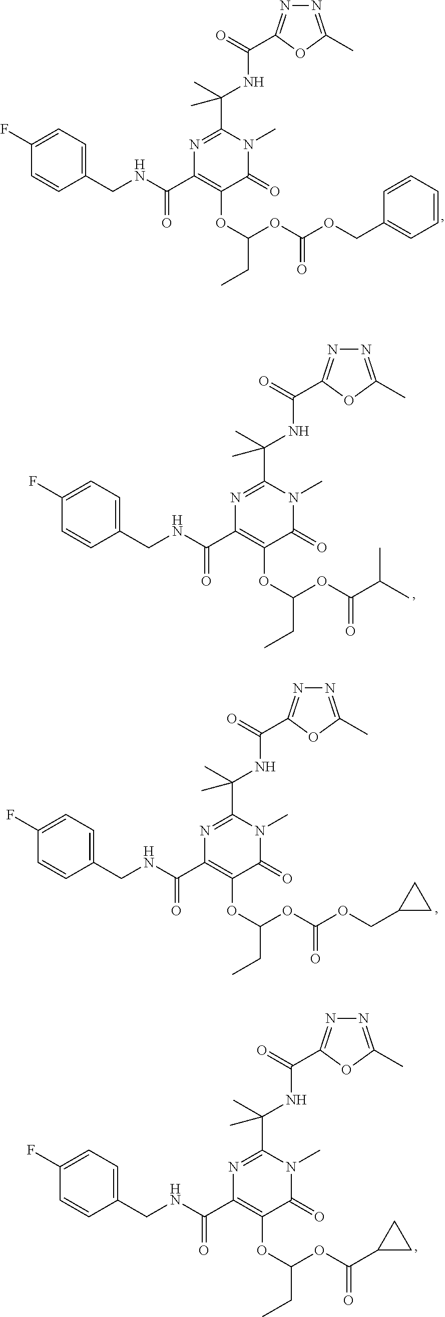 [SMILES: CCC(OC(=O)OCc1ccccc1)Oc1c(C(=O)NCc2ccc(F)cc2)nc(C(C)(C)NC(=O)c2nnc(C)o2)n(C)c1=O ; CCC(OC(=O)C(C)C)Oc1c(C(=O)NCc2ccc(F)cc2)nc(C(C)(C)NC(=O)c2nnc(C)o2)n(C)c1=O ; CCC(OC(=O)OCC1CC1)Oc1c(C(=O)NCc2ccc(F)cc2)nc(C(C)(C)NC(=O)c2nnc(C)o2)n(C)c1=O ; CCC(OC(=O)C1CC1)Oc1c(C(=O)NCc2ccc(F)cc2)nc(C(C)(C)NC(=O)c2nnc(C)o2)n(C)c1=O] 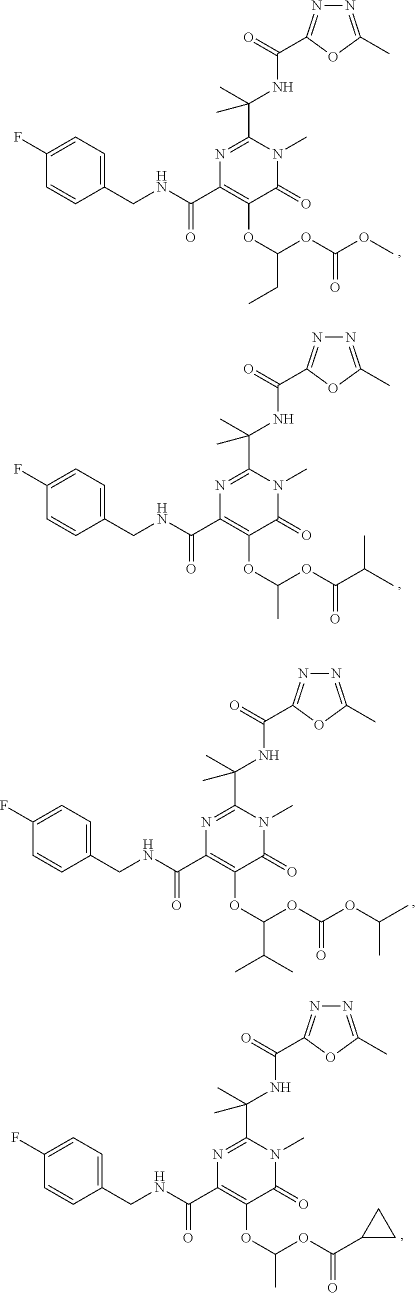 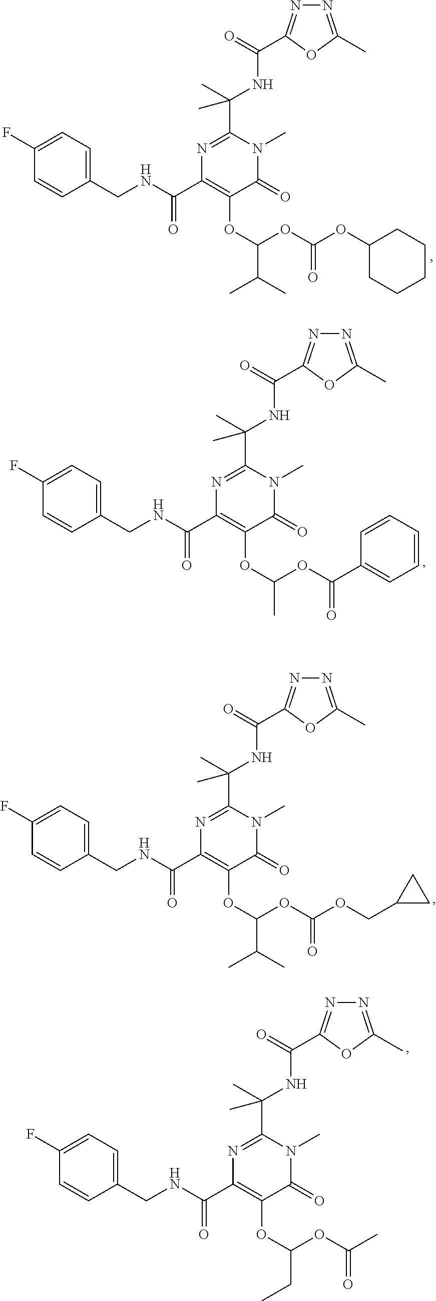 [SMILES: Cc1nnc(C(=O)NC(C)(C)c2nc(C(=O)NCc3ccc(F)cc3)c(OC(OC(=O)OC3CCCCC3)C(C)C)c(=O)n2C)o1 ; Cc1nnc(C(=O)NC(C)(C)c2nc(C(=O)NCc3ccc(F)cc3)c(OC(C)OC(=O)c3ccccc3)c(=O)n2C)o1 ; Cc1nnc(C(=O)NC(C)(C)c2nc(C(=O)NCc3ccc(F)cc3)c(OC(OC(=O)OCC3CC3)C(C)C)c(=O)n2C)o1 ; CCC(OC(C)=O)Oc1c(C(=O)NCc2ccc(F)cc2)nc(C(C)(C)NC(=O)c2nnc(C)o2)n(C)c1=O] 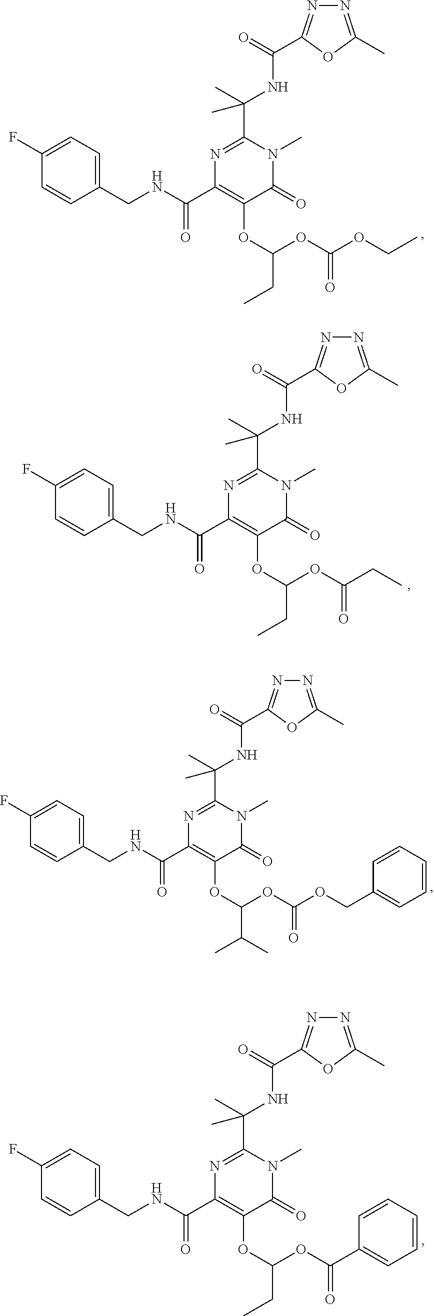 [SMILES: CCOC(=O)OC(CC)Oc1c(C(=O)NCc2ccc(F)cc2)nc(C(C)(C)NC(=O)c2nnc(C)o2)n(C)c1=O ; CCC(=O)OC(CC)Oc1c(C(=O)NCc2ccc(F)cc2)nc(C(C)(C)NC(=O)c2nnc(C)o2)n(C)c1=O ; Cc1nnc(C(=O)NC(C)(C)c2nc(C(=O)NCc3ccc(F)cc3)c(OC(OC(=O)OCc3ccccc3)C(C)C)c(=O)n2C)o1 ; CCC(OC(=O)c1ccccc1)Oc1c(C(=O)NCc2ccc(F)cc2)nc(C(C)(C)NC(=O)c2nnc(C)o2)n(C)c1=O] 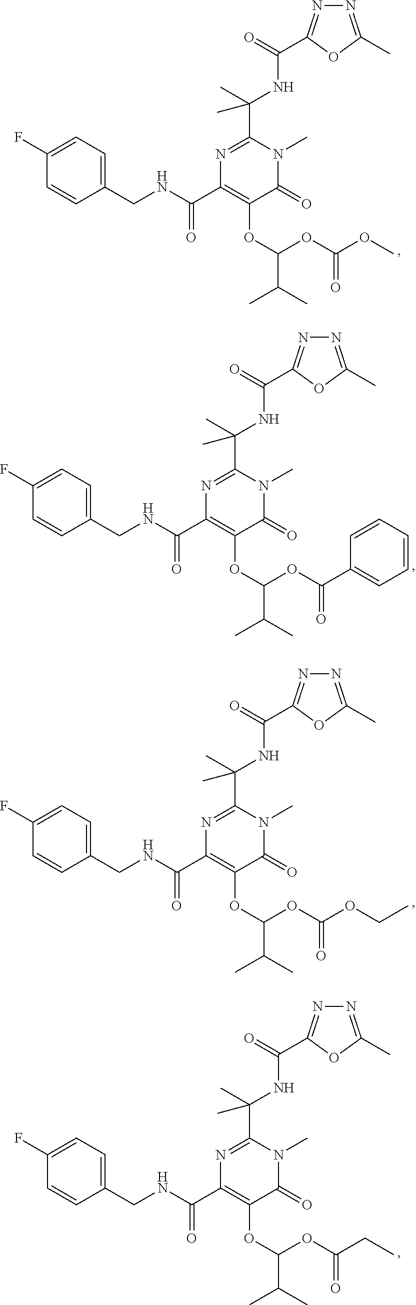 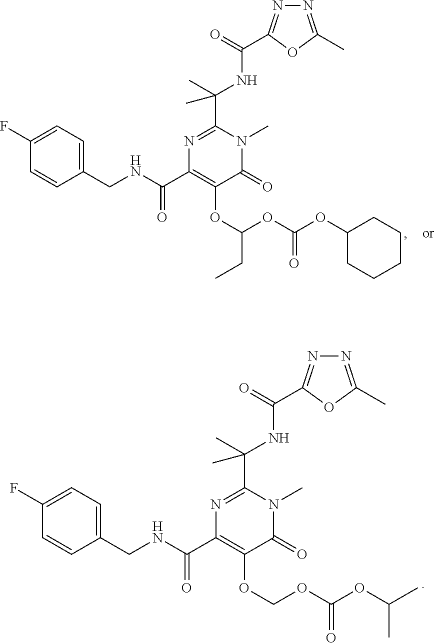 [SMILES: CCC(OC(=O)OC1CCCCC1)Oc1c(C(=O)NCc2ccc(F)cc2)nc(C(C)(C)NC(=O)c2nnc(C)o2)n(C)c1=O ; Cc1nnc(C(=O)NC(C)(C)c2nc(C(=O)NCc3ccc(F)cc3)c(OCOC(=O)OC(C)C)c(=O)n2C)o1]
or a pharmaceutically acceptable salt thereof.

14. A pharmaceutical composition comprising an effective amount of a compound according to claim 1 or a pharmaceutically acceptable salt thereof, and a pharmaceutically acceptable carrier.

15. A method for the inhibition of HIV integrase in a subject in need thereof which comprises administering to the subject an effective amount of the compound according to claim 1, or a pharmaceutically acceptable salt thereof.

16. A method for the treatment of infection by HIV or for the treatment, or delay in the onset or progression of AIDS in a subject in need thereof, which comprises administering to the subject an effective amount of the compound according to claim 1, or a pharmaceutically acceptable salt thereof.

17. (canceled)

18. (canceled)

19. The composition of claim 14, further comprising one or more additional therapeutic agents selected from raltegravir, lamivudine, abacavir, ritonavir, dolutegravir, arunavir, atazanavir, emtricitabine, tenofovir, elvitegravir, rilpivirine and lopinavir.

20. The method of claim 16, further comprising administering to the subject one or more additional therapeutic agents selected from raltegravir, abacavir, lamivudine, ritonavir and lopinavir, wherein the amounts administered of the compound of claim 1 and the one or more additional therapeutic agents, are together effective to treat infection by HIV or to treat, or delay the onset or progression of AIDS.
